(19) United States
(12) Patent Application Publication
(10) Pub. No.: US 2008/0038297 A1
(43) Pub. Date: Feb. 14, 2008

(54) GENETIC ADJUVANTS FOR VIRAL VACCINES

(76) Inventor: Kalpana GUPTA, New York, NY (US)

Correspondence Address: FROMMER LAWRENCE & HAUG, 745 FIFTH AVENUE- 10TH FL., NEW YORK, NY, 10151, US

(21) Appl. No.: 11/740,311

(22) Filed: Apr. 26, 2007

Related U.S. Application Data
(60) Provisional application No. 60/795,097, filed on Apr. 26, 2006.

(51) Int. Cl. A61K 48/00; A61K 31/7105; A61K 31/711; A61P 31/18; A61P 37/00; A61P 31/12; A61K 45/00

(52) U.S. Cl. 424/281.1; 424/93.2; 514/44

(57) ABSTRACT

The present invention relates to compositions and methods to improve expression of exogenous polypeptides, such as an antigen, epitope, immunogen, peptide or polypeptide of interest, in viral vaccines. More particularly, the present invention provides for viral vaccines with increased adjuvantation via a genetic adjuvant.

### INCORPORATION BY REFERENCE

This application claims benefit of U.S. provisional patent application Ser. No. 60/795,097 filed Apr. 26, 2006.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention.

### FIELD OF THE INVENTION

The present invention relates generally to viral vaccines and methods of using the same. More particularly, the present invention relates to viral vectors which may comprise one or more genetic adjuvants, resulting in enhanced immune response to an antigen expressed by a gene in a vector, advantageously a viral vector.

### BACKGROUND OF THE INVENTION

DNA vaccines, also referred to as genetic, plasmid or polynucleotide vaccines, represent a relatively simple and economical method to exploit gene transfer for immunization against antigens. The low toxicity associated with DNA vaccines favors its further development, but additional strategies to improve the potency of this approach are needed if it is to be successfully integrated into the clinical setting (reviewed by Shaw & Strong, Front Biosci. 2006 Jan. 1;11:11 89-98). DNA vaccination can overcome most disadvantages of conventional vaccine strategies and has potential for vaccines of the future. However, a commercial product still has not reached the market. One possible explanation could be the technique's failure to induce an efficient immune response in humans (reviewed by Glenting & Wessels, Microb Cell Fact. 2005 Sep. 6;4:26).

DNA vaccines and adeno-associated virus (“AAV”) vectors are similar in many respects. AAV vectors do not encode any viral genes, and the only viral sequences present in the AAV vector genome are the 145 nucleotide inverted terminal repeats (“ITRs”). However, AAV has the advantage of greatly increased in vivo transduction efficiency, due to specific delivery via the AAV capsid. Unlike DNA vaccines, humoral and cellular immune responses to AAV-expressed antigens can be elicited in nonhuman primates (“NHPs”) after a single AAV dose.

Genetic adjuvants for DNA vaccines have been reviewed (see, e.g, Calarota & Weiner, Expert Rev Vaccines. 2004 August ;3(4 Suppl):S135-49, Calarota & Weiner, Immunol Rev. 2004 June ;199:84-99 and Kutzler & Weiner, J Clin Invest. 2004 November ;114(9):1241-4), however genetic adjuvants for viral vaccines, especially for AAV-based viral vaccines, remain elusive.

There is a need for an effective and safe viral vaccine, especially with respect to expression of a target antigen, epitope, immunogen, peptide or polypeptide of interest in an amount sufficient to elicit a protective response.

Citation or identification of any document in this application is not an admission that such document is available as prior art to the present invention.

### SUMMARY OF THE INVENTION

The present invention relates to adjuvantation, or enhancement of immune responses, to the protein product of a gene expressed in a vector, preferably a viral vector, more preferably an AAV vector. Unlike many other vectors, such as Ad5, AAV induces only mild inflammatory response after injection into muscle, brain, liver, lung and retina, a desirable property of a gene therapy vector. However, it is possible that the inability to elicit innate immune responses after delivery serves to attenuate the immune response not only to the vector, but also to the protein product of the transgene. Therefore, AAV can be considered as a very safe, but poorly immunogenic delivery only vector. This suggests that AAV vectored vaccines might benefit from addition of a chemical or molecular adjuvant. The goal of such an adjuvant will be to create or mimic an inflammatory response coincident with expression of the transgene, to potentiate T and B cell responses against the gene product.

The biology lends itself to adjuvantation via a genetic adjuvant. While physical adjuvants may be effective, if transgene expression is delayed, the adjuvant may not be present to appropriately direct the immune response when expression is maximal. The result may be enhancing of undesirable anti-vector (input particle) immune responses, while having negligible effect on immune responses to payload antigen. The present invention seeks to enhance immune responses to the protein product of a pathogen gene expressed in an AAV vector, by co-expression of a second gene for an adjuvant.

The present invention relates to an immunogenic or vaccine composition which may comprise a vector, advantageously a viral vector, more advantageously an AAV vector, wherein the vector may comprise a polynucleotide sequence encoding a genetic adjuvant. In a preferred embodiment, the genetic adjuvant may be CTA1-DD, fas antigen, flagellin, IL-2 or IL-12.

The vector may further comprise a polynucleotide sequence encoding an antigen, epitope, immunogen, peptide or polypeptide of interest. Advantageously, the antigen, epitope, immunogen, peptide or polypeptide of interest is an immunodeficiency virus antigen, epitope, immunogen, peptide or polypeptide of interest. In a particularly preferred embodiment, the immunodeficiency virus antigen, epitope, immunogen, peptide or polypeptide of interest is an HIV or SIV antigen, epitope, immunogen, peptide or polypeptide of interest.

The invention also encompasses a formulation for delivery and expression of an antigen, epitope, immunogen, peptide or polypeptide of interest, wherein the formulation may comprise any one of the above-mentioned compositions and a pharmaceutically acceptable carrier, vehicle or excipient. In another embodiment, a formulation for delivery and expression of an antigen, epitope, immunogen, peptide or polypeptide of interest, wherein the formulation may comprise a vector encoding a genetic adjuvant, a vector comprising a polynucleotide sequence encoding an antigen, epitope, immunogen, peptide or polypeptide of interest and a pharmaceutically acceptable carrier, vehicle or excipient.

The present invention relates to methods of stimulating or eliciting an immune response in an animal which may comprise administering an effective amount of any of the herein-disclosed formulations to cells of the animal and expressing the antigen, epitope, immunogen, peptide or polypeptide of interest in the cells. Preferably, the animal is a human.

The invention also encompasses kits for performing any one of the methods of described above which may comprise the DNA plasmid or formulations of disclosed herein plus instructions for performing the methods of stimulating or eliciting an immune response in an animal.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings, in which:

FIG. 1 illustrates the effect of molecular adjuvants on immune responses to AAV vectored HIV vaccine;

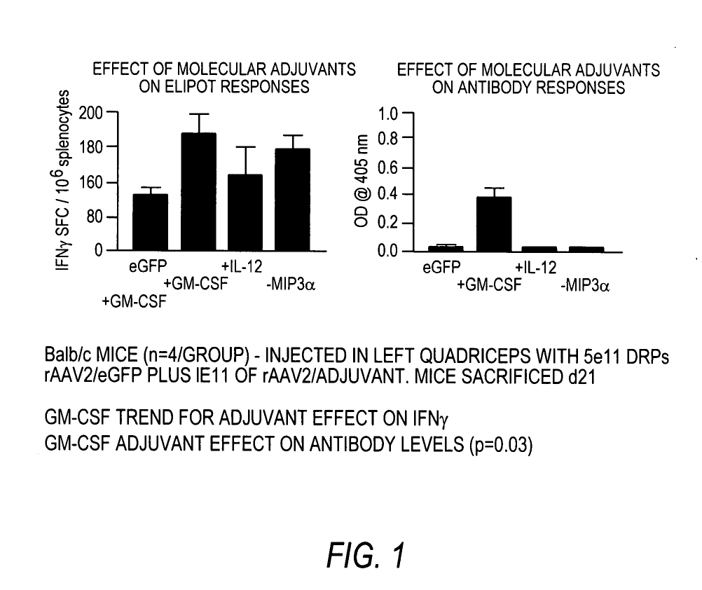

FIG. 2 illustrates sampling and assessment of immune response and efficacy;

FIG. 3 illustrates a representative AAV adjuvanted vector design;

FIG. 4A illustrates a parental vector clone expressing SIV gag-pro, clones expressing genetic adjuvant IL-2 and a polyprotein expressing SIV gag-pro and IL-2;

FIG. 4B illustrates western blots demonstrating the expression of SIV gag-pro and IL-2;

FIG. 5A illustrates SIV adjuvant constructs containing rhesus IL-12;

FIG. 5B illustrates western blots demonstrating gag and IL-12 expression;

FIG. 6A illustrates a cloning strategy for flagellin-SIVgag-pro fusion vectors;

FIG. 6B illustrates generated clones and predicted precursor proteins;

FIG. 6C illustrates western blots demonstrating gag and flagellin expression;

FIG. 7A illustrates additional flagellin constructs;

FIG. 7B illustrates western blots demonstrating gag and flagellin expression;

FIG. 8A illustrates CTA1-DD constructs and

FIG. 8B illustrates a western blot demonstrating CTA1-DD expression.

### DETAILED DESCRIPTION

The present invention relates to adjuvantation, or enhancement of immune responses, to the protein product of a gene expressed in a vector, preferably a viral vector, more preferably an AAV vector. AAV can be considered as a very safe, but poorly immunogenic delivery only vector. This suggests that AAV vectored vaccines might benefit from addition of a chemical or molecular adjuvant. While physical adjuvants may be effective, if transgene expression is delayed, the adjuvant may not be present to appropriately direct the immune response when expression is maximal. The result may be enhancing of undesirable anti-vector (input particle) immune responses, while having negligible effect on immune responses to payload antigen. The present invention seeks to enhance immune responses to the protein product of a pathogen gene expressed in a viral vector, by co-expression of a second gene for an adjuvant.

Although AAV vectors are preferred, the present invention contemplates other viral vectors. Additional viral vectors derived from viral families such as, but not limited to, Adenoviridae, Flaviviridae, Herpesviridae, Paramyxoviridae, Parvoviridae, Poxviridae and Reoviridae.

The present invention relates to an immunogenic or vaccine composition which may comprise a vector, advantageously a viral vector, more advantageously an AAV vector, wherein the vector may comprise a polynucleotide sequence encoding a genetic adjuvant. In a preferred embodiment, the genetic adjuvant may be CTA1-DD, fas antigen, flagellin, IL-2 or IL-12.

CTA1-DD is described in, for example, U.S. Pat. Nos. 6,589,529 and 5,917,026.

Fas antigen is described in, for example, U.S. Pat. Nos. 6,953,847; 6,949,360; 6,897,295; 6,855,543; 6,777,540; 6,465,618; 6,316,418; 6,306,820; 6,306,395; 6,284,801; 6,270,998; 6,177,592; 6,171,798; 6,114,507; 6,086,877; 6,054,436; 6,020,135; 6,015,559; 5,994,313; 5,874,546; 5,830,469; 5,663,070; 5,652,210 and 5,620,889.

In another embodiment, inducers of NK mediated apoptosis may be substituted or utilized in addition to fas antigen.

Flagellin is described in, for example, U.S. Pat. Nos. 6,805,865; 6,740,325; 6,585,980; 6,582,705; 6,419,932; 6,211,159; 6,130,082; 5,786,179; 5,750,115; 5,747,659; 5,725,858; 5,635,182; 5,153,312; 4,886,748 and 4,201,770.

IL-2 is described in, for example, U.S. Pat. Nos. 7,015,205; 6,994,976; 6,989,146; 6,977,072; 6,967,029; 6,962,694; 6,956,119; 6,955,807; 6,929,791; 6,921,530; 6,906,170; 6,905,680; 6,896,879; 6,893,869; 6,884,786; 6,884,598; 6,858,583; 6,852,313; 6,838,474; 6,828,147; 6,818,442; 6,774,226; 6,759,241; 6,756,038; 6,749,856; 6,746,669; 6,734,014; 6,719,972; 6,716,433; 6,713,279; 6,699,476; 6,693,083; 6,692,954; 6,682,909; 6,682,736; 6,660,723; 6,660,258; 6,627,647; 6,617,135; 6,613,762; 6,605,286; 6,605,273; 6,586,002; 6,559,137; 6,548,068; 6,534,277; 6,534,055; 6,531,453; 6,528,051; 6,525,102; 6,511,800; 6,509,313; 6,506,582; 6,503,713; 6,500,641; 6,497,876; 6,482,845; 6,479,258; 6,458,829; 6,455,503; 6,451,305; 6,448,073; 6,436,989; 6,413,771; 6,407,218; 6,406,710; 6,406,699; 6,406,696; 6,384,202; 6,383,739; 6,358,751; 6,358,524; 6,352,723; 6,348,449; 6,346,247; 6,323,027; 6,312,718; 6,291,483; 6,277,368; 6,274,552; 6,270,758; 6,267,955; 6,252,058; 6,251,866; 6,248,319; 6,232,087; 6,231,893; 6,218,371; 6,207,802; 6,207,454; 6,207,170; 6,197,925; 6,180,103; 6,168,787; 6,168,785; 6,166,186; 6,159,463; 6,159,462; 6,156,305; 6,150,099; 6,133,433; 6,127,170; 6,107,077; 6,099,847; 6,099,846; 6,093,723; 6,086,902; 6,083,503; 6,077,519; 6,070,126; 6,063,768; 6,063,375; 6,060,068; 6,054,297; 6,051,227; 6,048,530; 6,045,802; 6,017,544; 5,997,865; 5,989,546; 5,977,316; 5,976,522; 5,968,898; 5,965,366; 5,965,120; 5,961,979; 5,958,765; 5,932,427; 5,932,208; 5,919,480; 5,919,465; 5,897,990; 5,888,513; 5,879,673; 5,874,085; 5,874,076; 5,866,125; 5,858,978; 5,851,984; 5,847,004; 5,843,423; 5,843,397; 5,837,840; 5,814,314; 5,814,295; 5,800,810; 5,795,964; 5,789,185; 5,788,964; 5,783,557; 5,776,465; 5,756,540; 5,747,024; 5,698,530; 5,698,194; 5,693,322; 5,674,483; 5,650,152; 5,643,565; 5,635,478; 5,635,388; 5,635,386; 5,632,983; 5,627,052; 5,616,554; 5,591,632; 5,585,089; 5,580,561; 5,530,101; 5,503,841; 5,500,340; 5,474,899; 5,425,940; 5,409,698; 5,346,989; 5,250,295; 5,238,823; 5,225,535; 5,120,525; 5,100,664; 5,098,702; 4,971,795; RE33,252; 4,938,956; 4,894,227; 4,863,727; 4,863,726; 4,845,198; 4,604,377; 4,473,493 and 4,411,993.

IL-12 is described in, for example, U.S. Pat. Nos. 6,995,008; 6,989,146; 6,984,389; 6,956,119; 6,902,734; 6,899,885; 6,896,879; 6,893,869; 6,893,821; 6,867,000; 6,852,313; 6,838,290; 6,838,260; 6,830,751; 6,818,444; 6,774,226; 6,774,130; 6,759,241; 6,756,038; 6,749,856; 6,746,669; 6,716,433; 6,716,422; 6,713,279; 6,706,264; 6,693,105; 6,692,954; 6,682,909; 6,675,105; 6,660,258; 6,617,135; 6,605,286; 6,558,951; 6,548,068; 6,534,277; 6,528,051; 6,509,321; 6,503,713; 6,497,876; 6,479,258; 6,475,999; 6,455,503; 6,448,073; 6,423,308; 6,420,335; 6,407,218; 6,384,202; 6,384,018; 6,375,944; 6,365,165; 6,348,449; 6,346,247; 6,338,848; 6,274,552; 6,270,758; 6,239,116; 6,225,292; 6,225,117; 6,214,806; 6,207,454; 6,168,923; 6,168,787; 6,160,093; 6,159,462; 6,156,305; 6,127,170; 6,096,869; 6,080,742; 6,080,399; 6,077,519; 6,071,893; 6,070,126; 6,063,375; 6,051,227; 6,048,530; 6,045,802; 6,017,544; 6,004,812; 5,997,865; 5,985,264; 5,976,539; 5,962,424; 5,961,979; 5,919,480; 5,888,513; 5,851,984; 5,847,004; 5,843,423; 5,811,097; 5,756,540; 5,741,815; 5,736,524; 5,723,127; 5,705,151; 5,698,194; 5,693,322; 5,674,483; 5,665,347; 5,635,388; 5,632,983 and 5,547,852.

The genetic adjuvant of U.S. Pat. No. 6,693,086 may also be contemplated for the present invention. LT and CT genetic adjuvants are also useful in the present invention.

LT adjuvants are described in, for example, U.S. Pat. Nos. 6,987,176; 6,818,222; 6,589,529; 6,585,975; 6,576,757; 6,576,244; 6,569,435; 6,541,011; 6,440,423; 6,436,407; 6,413,523; 6,406,703; 6,129,923; 6,083,683; 6,077,678; 6,051,416; 6,033,673; 6,019,982; 5,985,243; 5,976,525; 5,919,463; 5,897,475; 5,869,066; 5,858,352; 5,681,736 and 5,679,564.

CT adjuvants are described in, for example, U.S. Pat. Nos. 6,849,725; 6,818,405; 6,797,471; 6,793,928; 6,759,200; 6,749,856; RE38,392; 6,607,732; 6,589,529; 6,565,828; 6,544,518; 6,472,585; 6,420,591; 6,395,964; 6,117,650; 6,074,352; 5,980,898; 5,917,026; 5,859,018; 5,783,182; 5,723,585; 5,679,545; 5,571,893; 5,565,215; 4,869,247; 4,411,888 and 4,034,090.

For purposes of the present invention, the only requirement for a genetic adjuvant is that it is encoded by a nucleotide sequence and expressed within a viral vector. Factors to be considered with genetic adjuvants include, but are not limited to, the size of the adjuvant gene versus viral vector capacity, time and cost of vector construction and production and safety.

As used herein, a genetic adjuvant refers to any biologically active factor, such as acytokine, an interleukin, a chemokine, a ligand, and optimally combinations thereof, which is expressed by a vector, and which, when administered with the priming DNA vaccine encoding an antigen, enhances the antigen-specific mucosal immune response compared with the immune response generated upon priming with the DNA vaccine encoding the antigen only. Other desirable genetic adjuvants include, without limitation, the DNA sequences encoding GM-CSF, the interferons (IFNs) (for example, IFN-α, IFN-β, and IFN-γ), the interleukins (ILs) (for example, IL-1β, IL-10, IL-12, IL-13), TNF-α, and combinations thereof. The genetic adjuvants may also be immunostimulatory polypeptide from Parapox virus, such as a polypeptide of Parapox virus strain D1701 or NZ2 or Parapox immunostimulatory polypeptides B2WL or PP30 (see, e.g., U.S. Pat. No. 6,752,995). Still other such biologically active factors that enhance the antigen-specific immune response may be readily selected by one of skill in the art, and a suitable plasmid vector containing the same factors constructed by known techniques.

The invention further provides for supplementing an immune response with physical adjuvants. Administration of a physical adjuvant is well known to one of skill in the art and may be co-administered and/or sequentially administered with the genetic adjuvant. Administration of the physical adjuvant may require routine experimentation which is within the purview of one of ordinary skill in the art. Factors to be considered with physical adjuvants include, but are not limited to, compatibility with live virus, timing of administration relative to onset of transgene expression and safety and feasibility. Accordingly, clinical stage adjuvants are preferred. Preferred physical adjuvants include, but are not limited to, CpG, CRONY (NKT cell CD1 ligand), IC31, Imiquimod, IQM and QS-21.

CpG is described in, for example, U.S. Pat. Nos. 7,014,992; 7,010,610; 6,994,870; 6,989,442; 6,979,728; 6,977,146; 6,977,069; 6,965,454; 6,964,951; 6,960,436; 6,960,434; 6,951,651; 6,949,520; 6,949,361; 6,942,972; 6,936,255; 6,932,972; 6,919,204; 6,914,148; 6,913,890; 6,911,306; 6,908,901; 6,893,820; 6,884,435; 6,881,561; 6,881,556; 6,878,616; 6,872,524; 6,858,388; 6,846,477; 6,835,541; 6,828,435; 6,821,957; 6,818,404; 6,815,429; 6,815,166; 6,811,982; 6,808,908; 6,794,137; 6,787,524; 6,785,741; 6,783,933; 6,773,897; 6,767,991; 6,762,281; 6,756,200; 6,753,015; 6,737,066; 6,733,777; 6,713,279; 6,709,818; 6,696,555; 6,693,086; 6,689,606; 6,689,588; 6,673,912; 6,671,845; 6,667,174; 6,653,295; 6,653,292; 6,649,751; 6,639,062; 6,636,623; 6,627,198; 6,613,894; 6,613,751; 6,605,432; 6,600,032; 6,599,700; 6,593,466; 6,590,092; 6,589,529; 6,576,752; 6,569,621; 6,566,338; 6,559,279; 6,552,006; 6,534,646; 6,534,523; 6,511,808; 6,486,132; 6,479,258; 6,476,000; 6,472,153; 6,465,438; 6,451,320; 6,429,199; 6,426,334; 6,420,380; 6,410,531; 6,406,705; 6,395,278; 6,366,793; 6,339,068; 6,331,393; 6,329,417; 6,329,379; 6,326,487; 6,323,180; 6,313,095; 6,309,828; 6,265,171; 6,251,594; 6,245,736; 6,239,116; 6,225,292; 6,218,371; 6,214,806; 6,214,556; 6,207,646; 6,200,756; 6,194,388; 6,191,306; 6,184,211; 6,180,614; 6,175,002; 6,153,591; 6,147,200; 6,122,671; 6,096,712; 6,090,791; 6,084,102; 6,057,465; 6,017,704; 6,004,750; 5,990,159; 5,972,883; 5,945,413; 5,942,610; 5,935,932; 5,917,122; 5,916,750; 5,863,901; 5,856,462; 5,851,762; 5,849,863; 5,840,879; 5,840,497; 5,834,431; 5,832,382; 5,786,146; 5,780,448; 5,736,626; 5,736,480; 5,700,926; 5,648,336; 5,554,744; 5,552,471; 5,532,170; 5,508,164; 5,472,672; 5,451,463; 5,419,966; 5,405,990; 5,401,837; 5,244,655; 5,114,918; 5,013,830; 4,945,059; 4,840,935; 4,569,917; 4,485,038; 4,431,654 and 4,368,034)

IC31 is described in, for example, U.S. Pat. No. 6,136,309. Imiquimod is described in, for example, U.S. Pat. Nos. 6,011,055 and 5,750,495. IQM is described in, for example, U.S. Pat. Nos. 6,465,173; 6,153,408; 6,011,146; 5,976,551 and 5,068,177. QS-21 is described in, for example, U.S. Pat. Nos. 7,014,856; 7,001,601; 6,979,448; 6,967,022; 6,936,253; 6,916,476; 6,905,686; 6,899,885; 6,890,535; 6,875,434; 6,855,316; 6,682,909; 6,645,495; 6,610,659; 6,589,529; 6,524,584; 6,458,369; 6,455,503; 6,403,104; 6,375,945; 6,355,256; 6,270,800; 6,248,585; 6,231,859; 6,123,948; 6,036,959; 5,723,130 and 5,612,030.

Other examples of known suitable adjuvants that can be used in the present invention include, but are not necessarily limited to, alum, aluminum phosphate, aluminum hydroxide, MF59 (4.3% w/v squalene, 0.5% w/v Tween 80, 0.5% w/v Span 85), CpG-containing nucleic acid (where the cytosine is unmethylated), QS21, MPL, 3DMPL, extracts from Aquilla, ISCOMS, LT/CT mutants, poly(D,L-lactide-co-glycolide) (PLG) microparticles, Quil A, interleukins, other Toll-like receptor ligands or NK cell ligands and the like alone or in combination. For experimental animals, one can use Freund's, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanine-2-(1′-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion. The effectiveness of an adjuvant may be determined by measuring the amount of antibodies directed against the immunogenic antigen.

Further exemplary adjuvants to enhance effectiveness of the composition include, but are not limited to: (1) oil-in-water emulsion formulations (with or without other specific immunostimulating agents such as muramyl peptides (see below) or bacterial cell wall components), such as for example (a) MF59.™. (W090/14837; Chapter 10 in Vaccine design: the subunit and adjuvant approach, eds. Powell & Newman, Plenum Press 1995), containing 5% Squalene, 0.5% Tween 80, and 0.5% Span 85 (optionally containing MTP-PE) formulated into submicron particles using a microfluidizer, (b) SAF, containing 10% Squalane, 0.4% Tween 80, 5% pluronic-blocked polymer L121, and thr-MDP either microfluidized into a submicron emulsion or vortexed to generate a larger particle size emulsion, and (c) RIBI.™. adjuvant system (RAS), (Ribi Immunochem, Hamilton, Mont.) containing 2% Squalene, 0.2% Tween 80, and one or more bacterial cell wall components such as monophosphorylipid A (MPL), trebalose dimycolate (TDM), and cell wall skeleton (CWS), preferably MPL+CWS DETOX.™.); (2) saponin adjuvants, such as QS21 or STIMULON.™. (Cambridge Bioscience, Worcester, Mass.) may be used or particles generated therefrom such as ISCOMs (immunostimulating complexes), which ISCOMS may be devoid of additional detergent e.g. WO00/07621; (3) Complete Freund's Adjuvant (CFA) and Incomplete Freund's Adjuvant (IFA); (4) cytokines, such as interleukins (e.g. IL-1, IL-2, IL-4, IL-5, IL-6, IL-7, IL-12 (WO99/44636), etc.), interferons (e.g. gamma interferon), macrophage colony stimulating factor (M-CSF), tumor necrosis factor (TNF), etc.; (5) monophosphoryl lipid A (MPL) or 3-O-deacylated MPL (3 dMPL) e.g. GB-2220221, EP-A-0689454, optionally in the substantial absence of alum when used with pneumococcal saccharides e.g. WO00/56358; (6) combinations of 3 dMPL with, for example, QS21 and/oroil-in-water emulsions e.g. EP-A-0835318, EP-A-0735898, EP-A-0761231; (7) oligonucleotides comprising CpG motif [Krieg Vaccine 2000, 19,618-622; Krieg Curr opin Mol Ther 2001 3:15-24; Roman et al., Nat. Med. 1997, 3,849-854; Weiner et al., PNAS USA, 1997, 94, 10833-10837; Davis et al, J. Immunol, 1998, 160, 870-876; Chu et al., J. Exp. Med, 1997, 186, 1623-1631; Lipford et al, Ear. J. Immunol., 1997, 27, 2340-2344; Moldoveami et al., Vaccine, 1988, 16, 1216-1224, Krieg et al., Nature, 1995, 374, 546-549; Klinman et al., PNAS USA, 1996, 93, 2879-2883; Ballas et al, J. Immunol, 1996, 157, 1840-1845; Cowdery et al, J. Immunol, 1996, 156, 4570-4575; Halpern et al, Cell Immunol, 1996, 167, 72-78; Yamamoto et al, Jpn. J. Cancer Res., 1988, 79, 866-873; Stacey et al, J. Immunol., 1996, 157,2116-2122; Messina et al, J. Immunol, 1991, 147, 1759-1764; Yi et al, J. Immunol, 1996, 157,4918-4925; Yi et al, J. Immunol, 1996, 157, 5394-5402; Yi et al, J. Immunol, 1998, 160, 4755-4761; and Yi et al, J. Immunol, 1998, 160, 5898-5906; International patent applications WO96/02555, WO98/16247, WO98/18810, WO98/40100, WO98/55495, WO98/37919 and WO98/52581] i.e. containing at least one CG dinucleotide, where the cytosine is unmethylated; (8) a polyoxyethylene ether or a polyoxyetbylene ester e.g. WO99/52549; (9) a polyoxyethylene soibitan ester surfactant in combination with an octoxynol (WO00/21207) or a polyoxyethylene alkyl ether or ester surfactant in combination with at least one additional non-ionic surfactant such as an octoxynol (WO01/21152); (10) a saponin and an immunostimulatory oligonucleotide (e.g. a CpG oligonucleotide) (WO00/62800); (11) an immunostimulant and a particle of metal salt e.g. WO00/23105; (12) a saponin and an oil-in-water emulsion e.g. WO99/11241; (13) a saponin (e.g. QS21)+3 dMPL+IM2 (optionally+a sterol) e.g. WO98/57659; (14) other substances that act as immunostimulating agents to enhance the efficacy of the composition. Muramyl peptides include N-acetyl-muramyl-L-threonyl-D-isoglutamine (thr-MDP), N-25 acetyl-normuramyl-L-alanyl-D-isoglutamine (nor-MDP), N-acetylmuramyl-L-alanyl-D-isoglutaminyl-L-alanine-2-(1′-2′-dipalmitoyl-sn-glycero-3-hydroxyphosphoryloxy)-ethylamine MTP-PE), etc.

The vector may further comprise a polynucleotide sequence encoding an antigen, epitope, immunogen, peptide or polypeptide of interest. Advantageously, the antigen, epitope, immunogen, peptide or polypeptide of interest is an immunodeficiency virus antigen, epitope, immunogen, peptide or polypeptide of interest. In a particularly preferred embodiment, the immunodeficiency virus antigen, epitope, immunogen, peptide or polypeptide of interest is an HIV or SIV antigen, epitope, immunogen, peptide or polypeptide of interest.

HIV sequences are disclosed in, for example, U.S. Pat. Nos. 7,008,784; 7,001,759; 6,995,008; 6,994,969; 6,964,763; 6,958,211; 6,942,969; 6,933,286; 6,897,301; 6,890,908; 6,869,933; 6,869,759; 6,861,515; 6,841,657; 6,828,148; 6,824,866; 6,821,945; 6,818,740; 6,814,934; 6,790,941; 6,783,981; 6,747,126; 6,734,160; 6,723,558; 6,703,493; 6,699,985; 6,696,291; 6,686,333; 6,686,150; 6,680,025; 6,670,181; RE38,352; 6,664,041; 6,660,904; 6,653,081; 6,649,340; 6,642,367; 6,630,455; 6,613,530; 6,596,539; 6,593,124; 6,586,192; 6,585,979; 6,562,575; 6,541,248; 6,534,312; 6,531,587; 6,531,276; 6,531,123; 6,528,251; 6,521,739; 6,514,736; 6,503,705; 6,498,033; 6,498,025; 6,492,120; 6,492,110; 6,489,098; 6,482,928; 6,482,805; 6,471,956; 6,468,982; 6,461,567; 6,458,527; 6,448,014; 6,440,461; 6,432,631; 6,429,306; 6,429,290; 6,429,009; 6,419,931; 6,410,257; 6,407,078; 6,404,907; 6,399,307; 6,372,956; 6,372,425; 6,350,730; 6,335,017; 6,309,853; 6,303,317; 6,303,295; 6,291,227; 6,287,605; 6,277,634; 6,265,149; 6,258,599; 6,258,319; 6,248,574; 6,242,568; 6,242,187; 6,235,881; 6,235,479; 6,232,120; 6,225,067; 6,222,025; 6,221,661; 6,221,578; 6,214,982; 6,207,426; 6,204,253; 6,197,755; 6,197,583; 6,197,563; 6,197,499; 6,171,785; 6,168,953; 6,168,948; 6,166,197; 6,156,952; 6,149,910; 6,140,466; 6,127,155; 6,124,448; 6,124,439; 6,114,349; 6,114,141; 6,110,466; 6,107,078; 6,107,020; 6,090,392; 6,086,891; 6,063,608; 6,048,837; 6,043,347; 6,040,166; 6,037,165; 6,037,152; 6,033,902; 6,033,881; 6,027,884; 6,015,661; 6,013,432; 6,010,895; 6,008,343; 6,007,984; 6,004,806; 6,001,989; 6,001,968; 6,001,648; 5,998,193; 5,994,056; 5,993,819; 5,990,276; 5,985,641; 5,981,505; 5,981,276; 5,981,171; 5,981,167; 5,972,701; 5,968,730; 5,962,635; 5,962,428; 5,958,768; 5,955,268; 5,939,262; 5,935,810; 5,919,625; 5,888,767; 5,885,806; 5,883,081; 5,876,976; 5,874,087; 5,869,339; 5,866,701; 5,858,785; 5,858,732; 5,856,188; 5,856,086; 5,854,967; 5,853,716; 5,846,546; 5,843,752; 5,843,640; 5,837,464; 5,830,876; 5,830,650; 5,830,641; 5,820,865; 5,817,792; 5,817,637; 5,817,635; 5,814,458; 5,798,365; 5,798,208; 5,792,756; 5,792,459; 5,786,199; 5,773,573; 5,773,260; 5,770,428; 5,767,233; 5,763,268; 5,747,292; 5,741,492; 5,739,118; 5,711,947; 5,698,687; 5,688,688; 5,683,661; 5,677,124; 5,672,695; 5,665,577; 5,654,195; 5,650,309; 5,650,302; 5,650,268; 5,639,600; 5,631,128; 5,618,664; 5,605,689; 5,594,123; 5,593,972; 5,587,285; 5,583,035; 5,571,712; 5,536,648; 5,532,146; 5,527,895; 5,527,673; 5,512,430; 5,503,721; 5,470,730; 5,439,809; 5,427,929; H001,431; 5,386,022; 5,352,600; 5,318,979; 5,314,809; 5,298,612; 5,278,173; 5,252,477; 5,234,809; 5,225,347; 5,221,608; 5,198,346; 5,184,020; 5,156,949; 5,153,202; 5,139,940; 5,110,802; 5,096,815; 5,079,352; 5,066,782; 5,030,449; 5,008,182; 4,965,188; 4,918,166 and 4,889,818.

In an advantageous embodiment, the immunodeficiency virus antigen, epitope, immunogen, peptide or polypeptide of interest of the present invention are HIV-1 proteins, advantageously HIV-1 proteins encoded by the env, gag, nef, reverse transcriptase (RT), protease (PR), integrase (IN), tat and rev genes, or any immunogenic fragment thereof. In an advantageous embodiment, env and RT sequences are derived from GenBank Accession No. AF067158 (see, e.g., Lole et al., J Virol. 1999 January ;73(1):152-60, the disclosure of which is incorporated by reference), gag and tat sequences are derived from GenBank Accession No. AF067157 (see, e.g., Lole et al., J Virol. 1999 January ;73(1):152-60, the disclosure of which is incorporated by reference), and rev and nef sequences are derived from GenBank Accession No. AF067154 (see, e.g., Lole et al., J Virol. 1999 January ;73(1):152-60, the disclosure of which is incorporated by reference).

SIV sequences are disclosed in, for example, U.S. Pat. Nos. 6,933,377; 6,841,657; 6,818,740; 6,790,657; 6,747,126; 6,712,612; 6,656,706; 6,596,539; 6,541,009; 6,531,123; 6,248,574; 6,083,504; 6,008,044; 5,777,074; 5,773,573; 5,753,674; 5,665,362; 5,654,195; 5,652,260 and 5,459,060.

In a particularly advantageous embodiment, the vector is an AAV vector comprising SIV and/or HIV and a molecular adjuvant. See, e.g., Example 3 for a schematic diagram of a rAAV vector encoding SIV gag-pro and a molecular adjuvant.

As used herein, the term “antigen” or “immunogen” means a substance that induces a specific immune response in a host animal. The antigen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a polypeptide, a peptide, an epitope, a hapten, or any combination thereof. Alternately, the immunogen or antigen may comprise a toxin or antitoxin.

The term “immunogenic protein or peptide” as used herein also refers includes peptides and polypeptides that are immunologically active in the sense that once administered to the host, it is able to evoke an immune response of the humoral and/or cellular type directed against the protein. Preferably the protein fragment is such that it has substantially the same immunological activity as the total protein. Thus, a protein fragment according to the invention comprises or consists essentially of or consists of at least one epitope or antigenic determinant. The term epitope relates to a protein site able to induce an immune reaction of the humoral type (B cells) and/or cellular type (T cells).

The term “immunogenic protein or peptide” further contemplates deletions, additions and substitutions to the sequence, so long as the polypeptide functions to produce an immunological response as defined herein. In this regard, particularly preferred substitutions will generally be conservative in nature, i. e., those substitutions that take place within a family of amino acids. For example, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cystine, serine threonine, tyrosine. Phenylalanine, tryptophan, and tyrosine are sometimes classified as aromatic amino acids. It is reasonably predictable that an isolated replacement of leucine with isoleucine or valine, or vice versa; an aspartate with a glutamate or vice versa; a threonine with a serine or vice versa; or a similar conservative replacement of an amino acid with a structurally related amino acid, will not have a major effect on the biological activity. Proteins having substantially the same amino acid sequence as the reference molecule but possessing minor amino acid substitutions that do not substantially affect the immunogenicity of the protein are, therefore, within the definition of the reference polypeptide.

The term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to a composition or vaccine of interest. Usually, an “immunological response” includes but is not limited to one or more of the following effects: the production of antibodies, B cells, helper T cells and/or cytotoxic T cells, directed specifically to an antigen or antigens included in the composition or vaccine of interest. Preferably, the host will display either a therapeutic or protective immunological response such that resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

The terms “immunogenic” protein or polypeptide as used herein also refers to an amino acid sequence which elicits an immunological response as described above. An “immunogenic” protein or polypeptide, as used herein, includes the full-length sequence of the protein, analogs thereof, or immunogenic fragments thereof. By “immunogenic fragment” is meant a fragment of a protein which includes one or more epitopes and thus elicits the immunological response described above. Such fragments can be identified using any number of epitope mapping techniques, well known in the art. See, e.g., Epitope Mapping Protocols in Methods in Molecular Biology, Vol. 66 (Glenn E. Morris, Ed., 1996) Humana Press, Totowa, N.J. For example, linear epitopes may be determined by e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad. Sci. USA 81:3998-4002; Geysen et al. (1986) Molec. Immunol. 23:709-715, all incorporated herein by reference in their entireties. Similarly, conformational epitopes are readily identified by determining spatial conformation of amino acids such as by, e.g., x-ray crystallography and 2-dimensional nuclear magnetic resonance. See, e.g., Epitope Mapping Protocols, supra. Methods especially applicable to the proteins of T. parva are fully described in the PCT Application Serial No. PCT/US2004/022605 incorporated herein by reference in its entirety.

Synthetic antigens are also included within the definition, for example, polyepitopes, flanking epitopes, and other recombinant or synthetically derived antigens. See, e.g., Bergmann et al. (1993) Eur. J. Immunol. 23:2777-2781; Bergmann et al. (1996) J. Immunol. 157:3242-3249; Suhrbier, A. (1997) Immunol. and Cell Biol. 75:402-408; Gardner et al. (1998) 12th World AIDS Conference, Geneva, Switzerland, Jun. 28-Jul. 3, 1998. Immunogenic fragments, for purposes of the present invention, will usually include at least about 3 amino acids, preferably at least about 5 amino acids, more preferably at least about 10-15 amino acids, and most preferably 25 or more amino acids, of the molecule. There is no critical upper limit to the length of the fragment, which could comprise nearly the full-length of the protein sequence, or even a fusion protein comprising at least one epitope of the protein.

Accordingly, a minimum structure of a polynucleotide expressing an epitope is that it comprises or consists essentially of or consists of nucleotides to encode an epitope or antigenic determinant. A polynucleotide encoding a fragment of the total protein or polyprotein, more advantageously, comprises or consists essentially of or consists of a minimum of 21 nucleotides, advantageously at least 42 nucleotides, and preferably at least 57, 87 or 150 consecutive or contiguous nucleotides of the sequence encoding the total protein or polyprotein. Epitope determination procedures, such as, generating overlapping peptide libraries (Hemmer B. et al., Immunology Today, 1998, 19 (4), 163-168), Pepscan (Geysen et al., (1984) Proc. Nat. Acad. Sci. USA, 81, 3998-4002; Geysen et al., (1985) Proc. Nat. Acad. Sci. USA, 82, 178-182; Van der Zee R. et al., (1989) Eur. J. Immunol., 19, 43-47; Geysen H. M., (1990) Southeast Asian J. Trop. Med. Public Health, 21, 523-533; Multipin.®. Peptide Synthesis Kits de Chiron) and algorithms (De Groot A. et al., (1999) Nature Biotechnology, 17, 533-561), and in PCT Application Serial No. PCT/US2004/022605 all of which are incorporated herein by reference in their entireties, can be used in the practice of the invention, without undue experimentation. Other documents cited and incorporated herein may also be consulted for methods for determining epitopes of an immunogen or antigen and thus nucleic acid molecules that encode such epitopes.

A “polynucleotide” is a polymeric form of nucleotides of any length, which contain deoxyribonucleotides, ribonucleotides, and analogs in any combination. Polynucleotides may have three-dimensional structure, and may perform any function, known or unknown. The term “polynucleotide” includes double-, single-stranded, and triple-helical molecules. Unless otherwise specified or required, any embodiment of the invention described herein that is a polynucleotide encompasses both the double stranded form and each of two complementary forms known or predicted to make up the double stranded form of either the DNA, RNA or hybrid molecule.

The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracyl, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism.

The invention further comprises a complementary strand to a polynucleotide encoding an antigen, epitope, immunogen, peptide or polypeptide of interest. The complementary strand can be polymeric and of any length, and can contain deoxyribonucleotides, ribonucleotides, and analogs in any combination.

The terms “protein”, “peptide”, “polypeptide” and “polypeptide fragment” are used interchangeably herein to refer to polymers of amino acid residues of any length. The polymer can be linear or branched, it may comprise modified amino acids or amino acid analogs, and it may be interrupted by chemical moieties other than amino acids. The terms also encompass an amino acid polymer that has been modified naturally or by intervention; for example disulfide bond formation, glycosylation, lipidation, acetylation, phosphorylation, or any other manipulation or modification, such as conjugation with a labeling or bioactive component.

An “isolated” polynucleotide or polypeptide is one that is substantially free of the materials with which it is associated in its native environment. By substantially free, is meant at least 50%, advantageously at least 70%, more advantageously at least 80%, and even more advantageously at least 90% free of these materials.

Hybridization reactions can be performed under conditions of different “stringency.” Conditions that increase stringency of a hybridization reaction are well known. See for example, “Molecular Cloning: A Laboratory Manual”, second edition (Sambrook et al. 1989). Examples of relevant conditions include (in order of increasing stringency): incubation temperatures of 25° C., 37° C., 50° C., and 68° C.; buffer concentrations of 10×SSC, 6×SSC, 1×SSC, 0.1×SSC (where SSC is 0.15 M NaCl and 15 mM citrate buffer) and their equivalent using other buffer systems; formamide concentrations of 0%, 25%, 50%, and 75%; incubation times from 5 minutes to 24 hours; 1, 2 or more washing steps; wash incubation times of 1, 2, or 15 minutes; and wash solutions of 6×SSC, 1×SSC, 0.1×SSC, or deionized water.

The invention further encompasses polynucleotides encoding functionally equivalent variants and derivatives of an antigen, epitope, immunogen, peptide or polypeptide of interest and functionally equivalent fragments thereof which may enhance, decrease or not significantly affect properties of the polypeptides encoded thereby. These functionally equivalent variants, derivatives, and fragments display the ability to retain antigenic activity. For instance, changes in a DNA sequence that do not change the encoded amino acid sequence, as well as those that result in conservative substitutions of amino acid residues, one or a few amino acid deletions or additions, and substitution of amino acid residues by amino acid analogs are those which will not significantly affect properties of the encoded polypeptide. Conservative amino acid substitutions are glycine/alanine; valine/isoleucine/leucine; asparagine/glutamine; aspartic acid/glutamic acid; serine/threonine/methionine; lysine/arginine; and phenylalanine/tyrosine/tryptophan. In one embodiment, the variants have at least 50%, at least 55%, at least 60%, at least 65%, at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98% or at least 99% homology or identity to the antigen, epitope, immunogen, peptide or polypeptide of interest.

For the purposes of the present invention, sequence identity or homology is determined by comparing the sequences when aligned so as to maximize overlap and identity while minimizing sequence gaps. In particular, sequence identity may be determined using any of a number of mathematical algorithms. A nonlimiting example of a mathematical algorithm used for comparison of two sequences is the algorithm of Karlin & Altschul, Proc. Natl. Acad. Sci. USA 1990; 87: 2264-2268, modified as in Karlin & Altschul, Proc. Natl. Acad. Sci. USA 1993;90: 5873-5877.

Another example of a mathematical algorithm used for comparison of sequences is the algorithm of Myers & Miller, CABIOS 1988;4: 11-17. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. Yet another useful algorithm for identifying regions of local sequence similarity and alignment is the FASTA algorithm as described in Pearson & Lipman, Proc. Natl. Acad. Sci. USA 1988; 85: 2444-2448.

Advantageous for use according to the present invention is the WU-BLAST (Washington University BLAST) version 2.0 software. WU-BLAST version 2.0 executable programs for several UNIX platforms can be downloaded from ftp ://blast.wustl.edu/blast/executables. This program is based on WU-BLAST version 1.4, which in turn is based on the public domain NCBI-BLAST version 1.4 (Altschul & Gish, 1996, Local alignment statistics, Doolittle ed., Methods in Enzymology 266: 460-480; Altschul et al., Journal of Molecular Biology 1990; 215: 403-410; Gish & States, 1993 ;Nature Genetics 3: 266-272; Karlin & Altschul, 1993;Proc. Natl. Acad. Sci. USA 90: 5873-5877; all of which are incorporated by reference herein).

In general, comparison of amino acid sequences is accomplished by aligning an amino acid sequence of a polypeptide of a known structure with the amino acid sequence of a the polypeptide of unknown structure. Amino acids in the sequences are then compared and groups of amino acids that are homologous are grouped together. This method detects conserved regions of the polypeptides and accounts for amino acid insertions and deletions. Homology between amino acid sequences can be determined by using commercially available algorithms (see also the description of homology above). In addition to those otherwise mentioned herein, mention is made too of the programs BLAST, gapped BLAST, BLASTN, BLASTP, and PSI-BLAST, provided by the National Center for Biotechnology Information. These programs are widely used in the art for this purpose and can align homologous regions of two amino acid sequences.

In all search programs in the suite the gapped alignment routines are integral to the database search itself. Gapping can be turned off if desired. The default penalty (Q) for a gap of length one is Q=9 for proteins and BLASTP, and Q=10 for BLASTN, but may be changed to any integer. The default per-residue penalty for extending a gap (R) is R=2 for proteins and BLASTP, and R=10 for BLASTN, but may be changed to any integer. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Alternatively or additionally, the term “homology” or “identity”, for instance, with respect to a nucleotide or amino acid sequence, can indicate a quantitative measure of homology between two sequences. The percent sequence homology can be calculated as (N_{ref}−N_{dif})*100/N_{ref}, wherein N_{dif} is the total number of non-identical residues in the two sequences when aligned and wherein N_{ref} is the number of residues in one of the sequences. Hence, the DNA sequence AGTCAGTC will have a sequence identity of 75% with the sequence AATCAATC (N_{ref}=8; N_{dif}=2).

Alternatively or additionally, “homology” or “identity” with respect to sequences can refer to the number of positions with identical nucleotides or amino acids divided by the number of nucleotides or amino acids in the shorter of the two sequences wherein alignment of the two sequences can be determined in accordance with the Wilbur and Lipman algorithm (Wilbur & Lipman, Proc Natl Acad Sci USA 1983; 80:726, incorporated herein by reference), for instance, using a window size of 20 nucleotides, a word length of 4 nucleotides, and a gap penalty of 4, and computer-assisted analysis and interpretation of the sequence data including alignment can be conveniently performed using commercially available programs (e.g., Intelligenetics™ Suite, Intelligenetics Inc. Calif.). When RNA sequences are said to be similar, or have a degree of sequence identity or homology with DNA sequences, thymidine (T) in the DNA sequence is considered equal to uracil (U) in the RNA sequence. Thus, RNA sequences are within the scope of the invention and can be derived from DNA sequences, by thymidine (T) in the DNA sequence being considered equal to uracil (U) in RNA sequences.

And, without undue experimentation, the skilled artisan can consult with many other programs or references for determining percent homology.

The invention further encompasses the polynucleotides encoding an antigen, epitope, immunogen, peptide or polypeptide of interest contained in a vector molecule or an expression vector and operably linked to a promoter element and optionally to an enhancer.

A “vector” refers to a recombinant DNA or RNA plasmid or virus that comprises a heterologous polynucleotide to be delivered to a target cell, either in vitro or in vivo. The heterologous polynucleotide may comprise a sequence of interest for purposes of therapy, and may optionally be in the form of an expression cassette. As used herein, a vector needs not be capable of replication in the ultimate target cell or subject. The term includes cloning vectors also included are viral vectors.

The term “recombinant” means a polynucleotide semisynthetic, or synthetic origin which either does not occur in nature or is linked to another polynucleotide in an arrangement not found in nature.

“Heterologous” means derived from a genetically distinct entity from the rest of the entity to which it is being compared. For example, a polynucleotide, may be placed by genetic engineering techniques into a plasmid or vector derived from a different source, and is a heterologous polynucleotide. A promoter removed from its native coding sequence and operatively linked to a coding sequence other than the native sequence is a heterologous promoter.

The polynucleotides of the invention may comprise additional sequences, such as additional encoding sequences within the same transcription unit, controlling elements such as promoters, ribosome binding sites, polyadenylation sites, additional transcription units under control of the same or a different promoter, sequences that permit cloning, expression, homologous recombination, and transformation of a host cell, and any such construct as may be desirable to provide embodiments of this invention.

Elements for the expression of an antigen, epitope, immunogen, peptide or polypeptide of interest are advantageously present in an inventive vector. In minimum manner, this comprises, consists essentially of, or consists of an initiation codon (ATG), a stop codon and a promoter, and optionally also a polyadenylation sequence for certain vectors such as plasmid and certain viral vectors, e.g., viral vectors other than poxviruses. When the polynucleotide encodes a polyprotein fragment advantageously, in the vector, an ATG is placed at 5′ of the reading frame and a stop codon is placed at 3′. Other elements for controlling expression may be present, such as enhancer sequences, stabilizing sequences, such as intron and signal sequences permitting the secretion of the protein.

Methods for making and/or administering a vector or recombinants or plasmid for expression of gene products of genes of the invention either in vivo or in vitro can be any desired method, e.g., a method which is by or analogous to the methods disclosed in, or disclosed in documents cited in: U.S. Pat. Nos. 4,603,112; 4,769,330; 4,394,448; 4,722,848; 4,745,051; 4,769,331; 4,945,050; 5,494,807; 5,514,375; 5,744,140; 5,744,141; 5,756,103; 5,762,938; 5,766,599; 5,990,091; 5,174,993; 5,505,941; 5,338,683; 5,494,807; 5,591,639; 5,589,466; 5,677,178; 5,591,439; 5,552,143; 5,580,859; 6,130,066; 6,004,777; 6,130,066; 6,497,883; 6,464,984; 6,451,770; 6,391,314; 6,387,376; 6,376,473; 6,368,603; 6,348,196; 6,306,400; 6,228,846; 6,221,362; 6,217,883; 6,207,166; 6,207,165; 6,159,477; 6,153,199; 6,090,393; 6,074,649; 6,045,803; 6,033,670; 6,485,729; 6,103,526; 6,224,882; 6,312,682; 6,348,450 and 6,312,683; U.S. patent application Ser. No. 920,197, filed Oct. 16, 1986; WO 90/01543; W091/11525; WO 94/16716; WO 96/39491; WO 98/33510; EP 265785; EP 0 370 573; Andreansky et al., Proc. Natl. Acad. Sci. USA 1996;93:11313-11318; Ballay et al., EMBO J. 1993;4:3861-65; Felgner et al., J. Biol. Chem. 1994;269:2550-2561; Frolov et al., Proc. Natl. Acad. Sci. USA 1996;93:11371-11377; Graham, Tibtech 1990;8:85-87; Grunhaus et al., Sem. Virol. 1992;3:237-52; Ju et al., Diabetologia 1998;41:736-739; Kitson et al., J. Virol. 1991;65:3068-3075; McClements et al., Proc. Natl. Acad. Sci. USA 1996;93:11414-11420; Moss, Proc. Natl. Acad. Sci. USA 1996;93:11341-11348; Paoletti, Proc. Natl. Acad. Sci. USA 1996;93:11349-11353; Pennock et al., Mol. Cell. Biol. 1984;4:399-406; Richardson (Ed), Methods in Molecular Biology 1995;39, “Baculovirus Expression Protocols,” Humana Press Inc.; Smith et al. (1983) Mol. Cell. Biol. 1983;3:2156-2165; Robertson et al., Proc. Natl. Acad. Sci. USA 1996;93:11334-11340; Robinson et al., Sem. Immunol. 1997;9:271; and Roizman, Proc. Natl. Acad. Sci. USA 1996;93:11307-11312. Thus, the vector in the invention can be any suitable recombinant virus or virus vector, such as a poxvirus (e.g., vaccinia virus, modified vaccinia—Ankara, avipox virus, canarypox virus, fowlpox virus, raccoonpox virus, swinepox virus, etc.), adenovirus (e.g., human adenovirus, chimpanzee adenovirus, canine adenovirus), herpesvirus (e.g. herpes simplex virus, Epstein-Barr virus, cytomegalovirus, canine herpesvirus), baculovirus, retrovirus, etc. (as in documents incorporated herein by reference); or the vector can be a plasmid. The herein cited and incorporated herein by reference documents, in addition to providing examples of vectors useful in the practice of the invention, can also provide sources for non-antigen, epitope, immunogen, peptide or polypeptide of interest or fragments thereof to be expressed by vector or vectors in, or included in, the compositions of the invention.

The present invention also relates to preparations comprising vectors, such as expression vectors, e.g., therapeutic compositions. The preparations can comprise, consist essentially of, or consist of one or more vectors, e.g., expression vectors, such as in vivo expression vectors, comprising, consisting essentially or consisting of (and advantageously expressing) one or more antigens, epitopes, immunogens peptides or polypeptides of interest. Advantageously, the vector contains and expresses a polynucleotide that includes, consists essentially of, or consists of a polynucleotide coding for (and advantageously expressing) an antigen, epitope, immunogen, peptide or polypeptide of interest, in a pharmaceutically acceptable carrier, excipient or vehicle. Thus, according to an embodiment of the invention, the other vector or vectors in the preparation comprises, consists essentially of or consists of a polynucleotide that encodes, and under appropriate circumstances the vector expresses one or more other proteins of antigen, epitope, immunogen, peptide or polypeptide of interest or a fragment thereof.

According to another embodiment, the vector or vectors in the preparation comprise, or consist essentially of, or consist of polynucleotide(s) encoding one or more proteins or fragment(s) thereof of antigen, epitope, immunogen, peptide or polypeptide of interest, the vector or vectors expressing the antigen, epitope, immunogen, peptide or polypeptide of interest. The inventive preparation advantageously comprises, consists essentially of, or consists of, at least two vectors comprising, consisting essentially of, or consisting of, and advantageously also expressing, advantageously in vivo under appropriate conditions or suitable conditions or in a suitable host cell, polynucleotides from different encoding the same proteins and/or for different proteins, but advantageously the same antigens, epitopes, immunogens, peptides or polypeptides of interest. Preparations containing one or more vectors containing, consisting essentially of or consisting of polynucleotides encoding, and advantageously expressing, advantageously in vivo, an antigen, epitope, immunogen, peptide or polypeptide of interest or fusion protein. The invention is also directed at mixtures of vectors that contain, consist essentially of, or consist of coding for, and express, different antigen, epitope, immunogen, peptide or polypeptide of interest.

According to one embodiment of the invention, the expression vector is a viral vector, in particular an in vivo expression vector. In an advantageous embodiment, the expression vector is an AAV vector. AAV is disclosed in, for example, U.S. Pat. Nos. 7,022,519; 7,015,026; 6,995,006; 6,989,264; 6,984,517; 6,979,539; 6,967,018; 6,953,690; 6,951,758; 6,951,753; 6,946,126; 6,943,019; 6,936,595; 6,936,466; 6,936,243; 6,933,373; 6,933,150; 6,933,113; 6,927,281; 6,924,128; 6,897,063; 6,893,865; 6,887,463; 6,855,314; 6,846,665; 6,841,357; 6,835,409; 6,821,775; 6,805,073; 6,797,702; 6,797,505; 6,793,926; 6,780,639; 6,780,409; 6,777,185; 6,764,845; 6,759,518; 6,759,237; 6,759,050; 6,743,423; 6,733,757; 6,723,558; 6,723,551; 6,723,512; 6,710,036; 6,703,237; 6,686,200; 6,677,155; 6,660,521; 6,656,727; 6,649,597; 6,642,051; 6,632,670; 6,627,617; 6,610,290; 6,607,882; 6,599,692; 6,596,535; 6,593,124; 6,593,123; 6,593,105; 6,589,523; 6,586,208; 6,582,692; 6,566,118; 6,558,948; 6,548,286; 6,541,258; 6,541,012; 6,534,261; 6,521,426; 6,521,225; 6,509,150; 6,506,600; 6,506,379; 6,503,888; 6,503,887; 6,503,717; 6,498,244; 6,491,907; 6,485,966; 6,482,634; 6,482,633; 6,475,769; 6,468,771; 6,468,524; 6,458,587; 6,451,594; 6,448,074; 6,440,742; 6,436,708; 6,429,001; 6,428,988; 6,416,992; 6,410,300; 6,391,858; 6,387,670; 6,387,368; 6,383,794; 6,376,237; 6,365,403; 6,346,415; 6,335,011; 6,329,181; 6,325,998; 6,312,957; 6,306,650; 6,303,371; 6,302,685; 6,294,379; 6,294,370; 6,287,857; 6,274,354; 6,270,996; 6,261,834; 6,261,551; 6,258,595; 6,251,677; 6,242,426; 6,232,105; 6,225,113; 6,221,646; 6,211,163; 6,207,457; 6,207,453; 6,171,597; 6,162,796; 6,156,303; 6,143,548; 6,117,680; 6,110,456; 6,093,570; 6,057,152; 6,040,183; 6,040,172; 6,037,177; 6,033,885; 6,027,931; 6,020,192; 6,004,797; 6,001,650; 5,976,853; 5,965,441; 5,962,424; 5,962,313; 5,958,768; 5,945,335; 5,942,496; 5,940,530; 5,939,538; 5,928,943; 5,874,556; 5,874,304; 5,871,982; 5,869,305; 5,869,230; 5,866,552; 5,863,541; 5,858,351; 5,856,152; 5,846,546; 5,846,528; 5,837,484; 5,834,440; 5,789,390; 5,780,280; 5,773,289; 5,763,416; 5,756,283; 5,753,500; 5,741,683; 5,691,176; 5,688,676; 5,688,675; 5,681,731; 5,677,158; 5,658,776; 5,650,309; 5,646,034; 5,622,856; 5,604,090; 5,478,745; 5,474,935; 5,436,146; 5,416,017; 5,354,678; 5,252,479; 5,173,414; 5,155,468; 5,139,941; 4,797,368 and 4,559,713.

In a less preferred embodiment, embodiment, the expression vector is an adenovirus vector. The adenovirus may be a human Ad5 vector, an E1-deleted and/or an E3-deleted adenovirus.

For information on the method to generate recombinants thereof and how to administer recombinants thereof, the skilled artisan can refer documents cited herein and to WO90/12882, e.g., as to vaccinia virus mention is made of U.S. Pat. Nos. 4,769,330, 4,722,848, 4,603,112, 5,110,587, 5,494,807, and 5,762,938 inter alia; as to fowlpox, mention is made of U.S. Pat. Nos. 5,174,993, 5,505,941 and U.S. Pat. No. 5,766,599 inter alia; as to canarypox mentionis made of U.S. Pat. No. 5,756,103 inter alia; as to swinepox mention is made of U.S. Pat. No. 5,382,425 inter alia; and, as to raccoonpox, mention is made of WO00/03030 inter alia.

When the expression vector is a vaccinia virus, insertion site or sites for the polynucleotide or polynucleotides to be expressed are advantageously at the thymidine kinase (TK) gene or insertion site, the hemagglutinin (HA) gene or insertion site, the region encoding the inclusion body of the A type (ATI); see also documents cited herein, especially those pertaining to vaccinia virus. In the case of canarypox, advantageously the insertion site or sites are ORF(s) C3, C5 and/or C6; see also documents cited herein, especially those pertaining to canarypox virus. In the case of fowlpox, advantageously the insertion site or sites are ORFs F7 and/or F8; see also documents cited herein, especially those pertaining to fowlpox virus. The insertion site or sites for MVA virus area advantageously as in various publications, including Carroll M. W. et al., Vaccine, 1997, 15 (4), 387-394; Stittelaar K. J. et al., J. Virol., 2000, 74 (9), 4236-4243; Sutter G. et al., 1994, Vaccine, 12 (11), 1032-1040; and, in this regard it is also noted that the complete MVA genome is described in Antoine G., Virology, 1998, 244, 365-396, which enables the skilled artisan to use other insertion sites or other promoters.

Advantageously, the polynucleotide to be expressed is inserted under the control of a cytomegalovirus (CMV) promoter. The CMV promoter may be a minimal or full-length promoter (see, e.g., U.S. Pat. No. 6,368,825). In some advantageous embodiments, the CMV promoter is a shortened or truncated promoter which permits the cloning of a larger genetic adjuvant into the vector.

Advantageously, the polynucleotide to be expressed is inserted under the control of a specific poxvirus promoter, e.g., the vaccinia promoter 7.5 kDa (Cochran et al., J. Virology, 1985, 54, 30-35), the vaccinia promoter I3L (Riviere et al., J. Virology, 1992, 66, 3424-3434), the vaccinia promoter HA (Shida, Virology, 1986, 150, 451-457), the cowpox promoter ATI (Funahashi et al., J. Gen. Virol., 1988, 69, 35-47), the vaccinia promoter H6 (Taylor J. et al., Vaccine, 1988, 6, 504-508; Guo P. et al. J. Virol., 1989, 63, 4189-4198; Perkus M. et al., J. Virol., 1989, 63, 3829-3836), inter alia.

In a particular embodiment the viral vector is an adenovirus, such as a human adenovirus (HAV) or a canine adenovirus (CAV).

In one embodiment the viral vector is a human adenovirus, in particular a serotype 5 adenovirus, rendered incompetent for replication by a deletion in the E1 region of the viral genome, in particular from about nucleotide 459 to about nucleotide 3510 by reference to the sequence of the hAd5 disclosed in Genbank under the accession number M73260 and in the referenced publication J. Chroboczek et al Virol. 1992, 186, 280-285. The deleted adenovirus is propagated in E1-expressing 293 (F. Graham et al J. Gen. Virol. 1977, 36, 59-72) or PER cells, in particular PER.C6 (F. Falloux et al Human Gene Therapy 1998, 9, 1909-1917). The human adenovirus can be deleted in the E3 region, in particular from about nucleotide 28592 to about nucleotide 30470. The deletion in the E1 region can be done in combination with a deletion in the E3 region (see, e.g. J. Shriver et al. Nature, 2002, 415, 331-335, F. Graham et al Methods in Molecular Biology Vol. 7: Gene Transfer and Expression Protocols Edited by E. Murray, The Human Press Inc, 1991, p 109-128; Y. Ilan et al Proc. Natl. Acad. Sci. 1997, 94, 2587-2592; U.S. Pat. No. 6,133,028; U.S. Pat. No. 6,692,956; S. Tripathy et al Proc. Natl. Acad. Sci. 1994, 91, 11557-11561; B. Tapnell Adv. Drug Deliv. Rev. 1993, 12, 185-199;X. Danthinne et al Gene Thrapy 2000, 7, 1707-1714; K. Berkner Bio Techniques 1988, 6, 616-629; K. Berkner et al Nucl. Acid Res. 1983, 11, 6003-6020; C. Chavier et al J. Virol. 1996, 70, 4805-4810). The insertion sites can be the E1 and/or E3 loci (region) eventually after a partial or complete deletion of the E1 and/or E3 regions. Advantageously, when the expression vector is an adenovirus, the polynucleotide to be expressed is inserted under the control of a promoter functional in eukaryotic cells, such as a strong promoter, preferably a cytomegalovirus immediate-early gene promoter (CMV-IE promoter), in particular the enhancer/promoter region from about nucleotide −734 to about nucleotide +7 in M. Boshart et al Cell 1985, 41, 521-530 or the enhancer/promoter region from the pCI vector from Promega Corp. The CMV-IE promoter is advantageously of murine or human origin. The promoter of the elongation factor 1α can also be used. A muscle specific promoter can also be used (X. Li et al Nat. Biotechnol. 1999, 17, 241-245). Strong promoters are also discussed herein in relation to plasmid vectors. In one embodiment, a splicing sequence can be located downstream of the enhancer/promoter region. For example, the intron 1 isolated from the CMV-IE gene (R. Stenberg et al J. Virol. 1984, 49, 190), the intron isolated from the rabbit or human β-globin gene, in particular the intron 2 from the b-globin gene, the intron isolated from the immunoglobulin gene, a splicing sequence from the SV40 early gene or the chimeric intron sequence isolated from the pCI vector from Promege Corp. comprising the human β-globin gene donor sequence fused to the mouse immunoglobulin acceptor sequence (from about nucleotide 890 to about nucleotide 1022 in Genbank under the accession number CVU47120). A poly(A) sequence and terminator sequence can be inserted downstream the polynucleotide to be expressed, e.g. a bovine growth hormone gene, in particular from about nucleotide 2339 to about nucleotide 2550 in Genbank under the accession number BOVGHRH, a rabbit β-globin gene or a SV40 late gene polyadenylation signal.

In another embodiment the viral vector is a canine adenovirus, in particular a CAV-2 (see, e.g. L. Fischer et al. Vaccine, 2002, 20, 3485-3497; U.S. Pat. No. 5,529,780; U.S. Pat. No. 5,688,920; PCT Application No. WO95/14102). For CAV, the insertion sites can be in the E3 region and/or in the region located between the E4 region and the right ITR region (see U.S. Pat. No. 6,090,393; U.S. Pat. No. 6,156,567). In one embodiment the insert is under the control of a promoter, such as a cytomegalovirus immediate-early gene promoter (CMV-IE promoter) or a promoter already described for a human adenovirus vector. A poly(A) sequence and terminator sequence can be inserted downstream the polynucleotide to be expressed, e.g. a bovine growth hormone gene or a rabbit β-globin gene polyadenylation signal.

In another particular embodiment the viral vector is a herpesvirus such as a canine herpesvirus (CHV) or a feline herpesvirus (FHV). For CHV, the insertion sites may be in particular in the thymidine kinase gene, in the ORF3, or in the UL43 ORF (see U.S. Pat. No. 6,159,477). In one embodiment the polynucleotide to be expressed is inserted under the control of a promoter functional in eukaryotic cells, advantageously a CMV-IE promoter (murine or human). A poly(A) sequence and terminator sequence can be inserted downstream the polynucleotide to be expressed, e.g. bovine growth hormone or a rabbit β-globin gene polyadenylation signal.

According to a yet further embodiment of the invention, the expression vector is a plasmid vector or a DNA plasmid vector, in particular an in vivo expression vector. In a specific, non-limiting example, the pVR1020 or 1012 plasmid (VICAL Inc.; Luke C. et al., Journal of Infectious Diseases, 1997, 175, 91-97; Hartikka J. et al., Human Gene Therapy, 1996, 7, 1205-1217, see, e.g., U.S. Pat. Nos. 5,846,946 and 6,451,769) can be utilized as a vector for the insertion of a polynucleotide sequence. The pVR1020 plasmid is derived from pVR1012 and contains the human tPA signal sequence. In one embodiment the human tPA signal comprises from amino acid M(1) to amino acid S(23) in Genbank under the accession number HUMTPA14. In another specific, non-limiting example, the plasmid utilized as a vector for the insertion of a polynucleotide sequence can contain the signal peptide sequence of equine IGF1 from amino acid M(24) to amino acid A(48) in Genbank under the accession number U28070. Additional information on DNA plasmids which may be consulted or employed in the practice are found, for example, in U.S. Pat. Nos. 6,852,705; 6,818,628; 6,586,412; 6,576,243; 6,558,674; 6,464,984; 6,451,770; 6,376,473 and 6,221,362.

The term plasmid covers any DNA transcription unit comprising a polynucleotide according to the invention and the elements necessary for its in vivo expression in a cell or cells of the desired host or target; and, in this regard, it is noted that a supercoiled or non-supercoiled, circular plasmid, as well as a linear form, are intended to be within the scope of the invention.

Each plasmid comprises or contains or consists essentially of, in addition to the polynucleotide encoding an antigen, epitope, immunogen, peptide or polypeptide of interest, optionally fused with a heterologous peptide sequence, variant, analog or fragment, operably linked to a promoter or under the control of a promoter or dependent upon a promoter. In general, it is advantageous to employ a strong promoter functional in eukaryotic cells. The preferred strong promoter is the immediate early cytomegalovirus promoter (CMV-IE) of human or murine origin, or optionally having another origin such as the rat or guinea pig. The CMV-IE promoter can comprise the actual promoter part, which may or may not be associated with the enhancer part. Reference can be made to EP-A-260 148, EP-A-323 597, U.S. Pat. Nos. 5,168,062, 5,385,839, and 4,968,615, as well as to PCT Application No WO87/03905. The CMV-IE promoter is advantageously a human CMV-IE (Boshart M. et al., Cell., 1985, 41, 521-530) or murine CMV-IE.

In more general terms, the promoter has either a viral or a cellular origin. A strong viral promoter other than CMV-IE that may be usefully employed in the practice of the invention is the early/late promoter of the SV40 virus or the LTR promoter of the Rous sarcoma virus. A strong cellular promoter that may be usefully employed in the practice of the invention is the promoter of a gene of the cytoskeleton, such as e.g. the desmin promoter (Kwissa M. et al., Vaccine, 2000, 18, 2337-2344), or the actin promoter (Miyazaki J. et al., Gene, 1989, 79, 269-277).

Functional sub fragments of these promoters, i.e., portions of these promoters that maintain an adequate promoting activity, are included within the present invention, e.g. truncated CMV-IE promoters according to PCT Application No. WO98/00166 or U.S. Pat. No. 6,156,567 can be used in the practice of the invention. A promoter in the practice of the invention consequently includes derivatives and sub fragments of a full-length promoter that maintain an adequate promoting activity and hence function as a promoter, preferably promoting activity substantially similar to that of the actual or full-length promoter from which the derivative or sub fragment is derived, e.g., akin to the activity of the truncated CMV-IE promoters of U.S. Pat. No. 6,156,567 to the activity of full-length CMV-IE promoters. Thus, a CMV-IE promoter in the practice of the invention can comprise or consist essentially of or consist of the promoter portion of the full-length promoter and/or the enhancer portion of the full-length promoter, as well as derivatives and sub fragments.

Preferably, the plasmids comprise or consist essentially of other expression control elements. It is particularly advantageous to incorporate stabilizing sequence(s), e.g., intron sequence(s), preferably the first intron of the hCMV-IE (PCT Application No. WO89/01036), the intron II of the rabbit β-globin gene (van Ooyen et al., Science, 1979, 206, 337-344).

As to the polyadenylation signal (polyA) for the plasmids and viral vectors other than poxviruses, use can more be made of the poly(A) signal of the bovine growth hormone (bGH) gene (see U.S. Pat. No. 5,122,458), or the poly(A) signal of the rabbit β-globin gene or the poly(A) signal of the SV40 virus.

According to another embodiment of the invention, the expression vectors are expression vectors used for the in vitro expression of proteins in an appropriate cell system. The expressed proteins can be harvested in or from the culture supernatant after, or not after secretion (if there is no secretion a cell lysis typically occurs or is performed), optionally concentrated by concentration methods such as ultrafiltration and/or purified by purification means, such as affinity, ion exchange or gel filtration-type chromatography methods.

A “host cell” denotes a prokaryotic or eukaryotic cell that has been genetically altered, or is capable of being genetically altered by administration of an exogenous polynucleotide, such as a recombinant plasmid or vector. When referring to genetically altered cells, the term refers both to the originally altered cell and to the progeny thereof. Advantageous host cells include, but are not limited to, baby hamster kidney (BHK) cells, colon carcinoma (Caco-2) cells, COS7 cells, MCF-7 cells, MCF-10A cells, Madin-Darby canine kidney (MDCK) lines, mink lung (Mv1Lu) cells, MRC-5 cells, U937 cells and VERO cells. Polynucleotides comprising a desired sequence can be inserted into a suitable cloning or expression vector, and the vector in turn can be introduced into a suitable host cell for replication and amplification. Polynucleotides can be introduced into host cells by any means known in the art. The vectors containing the polynucleotides of interest can be introduced into the host cell by any of a number of appropriate means, including direct uptake, endocytosis, transfection, f-mating, electroporation, transfection employing calcium chloride, rubidium chloride, calcium phosphate, DEAE-dextran, or other substances; microprojectile bombardment; lipofection; and infection (where the vector is infectious, for instance, a retroviral vector). The choice of introducing vectors or polynucleotides will often depend on features of the host cell.

In an advantageous embodiment, the invention provides for the administration of a therapeutically effective amount of a formulation for the delivery and expression of an antigen, epitope, immunogen, peptide or polypeptide of interest in a target cell. Determination of the therapeutically effective amount is routine experimentation for one of ordinary skill in the art. In one embodiment, the formulation comprises an expression vector comprising a polynucleotide that expresses an antigen, epitope, immunogen, peptide or polypeptide of interest and a pharmaceutically acceptable carrier, vehicle or excipient. In an advantageous embodiment, the pharmaceutically acceptable carrier, vehicle or excipient facilitates transfection and/or improves preservation of the vector or protein.

The pharmaceutically acceptable carriers or vehicles or excipients are well known to the one skilled in the art. For example, a pharmaceutically acceptable carrier or vehicle or excipient can be a 0.9% NaCl (e.g., saline) solution or a phosphate buffer. Other pharmaceutically acceptable carrier or vehicle or excipients that can be used for methods of this invention include, but are not limited to, poly-(L-glutamate) or polyvinylpyrrolidone. The pharmaceutically acceptable carrier or vehicle or excipients may be any compound or combination of compounds facilitating the administration of the vector (or protein expressed from an inventive vector in vitro); advantageously, the carrier, vehicle or excipient may facilitate transfection and/or improve preservation of the vector (or protein). Doses and dose volumes are herein discussed in the general description and can also be determined by the skilled artisan from this disclosure read in conjunction with the knowledge in the art, without any undue experimentation.

The antigens may be combined with conventional excipients, such as pharmaceutical grades of mannitol, lactose, starch, magnesium stearate, sodium saccharin, talcum, cellulose, glucose, sucrose, magnesium, carbonate, and the like. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of antigen in these formulations can vary widely, and will be selected primarily based on fluid volumes, viscosities, body weight and the like in accordance with the particular mode of administration selected and the patient's needs. The resulting compositions may be in the form of a solution, suspension, tablet, pill, capsule, powder, gel, cream, lotion, ointment, aerosol or the like.

The concentration of immunogenic antigens of the invention in the pharmaceutical formulations can vary widely, i.e. from less than about 0.1%, usually at or at least about 2% to as much as 20% to 50% or more by weight, and will be selected primarily by fluid volumes, viscosities, etc., in accordance with the particular mode of administration selected.

Advantageously, the pharmaceutical and/or therapeutic compositions and/or formulations according to the invention comprise or consist essentially of or consist of an effective quantity to elicit a therapeutic response of one or more expression vectors and/or polypeptides as discussed herein; and, an effective quantity can be determined from this disclosure, including the documents incorporated herein, and the knowledge in the art, without undue experimentation.

In the case of therapeutic and/or pharmaceutical compositions based on a plasmid vector, a dose can comprise, consist essentially of or consist of, in general terms, about in 1 μg to about 2000 μg, advantageously about 50 μg to about 1000 μg and more advantageously from about 100 μg to about 800 μg of plasmid expressing the antigen, epitope, immunogen, peptide or polypeptide of interest. When the therapeutic and/or pharmaceutical compositions based on a plasmid vector is administered with electroporation the dose of plasmid is generally between about 0.1 μg and 1 mg, advantageously between about 1 μg and 100 μg, advantageously between about 2 μg and 50 μg. The dose volumes can be between about 0.1 and about 2 ml, advantageously between about 0.2 and about 1 ml. These doses and dose volumes are suitable for the treatment of canines and other mammalian target species such as equines and felines.

The therapeutic and/or pharmaceutical composition contains per dose from about 10⁴ to about 10¹¹, advantageously from about 10⁵ to about 10¹⁰ and more advantageously from about 10⁶ to about 10⁹ viral particles of recombinant virus, advantageously AAV, expressing a genetic adjuvant and/or an antigen, epitope, immunogen, peptide or polypeptide of interest. In the case of therapeutic and/or pharmaceutical compositions based on a poxvirus, a dose can be between about 10² pfu and about 10⁹ pfu. The pharmaceutical composition contains per dose from about 10⁵ to 10⁹, advantageously from about 10⁶ to 10⁸ pfu of poxvirus or herpesvirus recombinant expressing a genetic adjuvant and/or an antigen, epitope, immunogen, peptide or polypeptide of interest.

The dose volume of compositions for target species that are mammals, e.g., the dose volume of canine compositions, based on viral vectors, e.g., non-poxvirus-viral-vector-based compositions, is generally between about 0.1 to about 2.0 ml, preferably between about 0.1 to about 1.0 ml, and more preferably between about 0.5 ml to about 1.0 ml.

With inactivated compositions of the virus or organism or pathogen produced on the new cell culture, the animal may be administered approximately 10⁴-10⁹ equivalent CCID₅₀ (titer before inactivation), advantageously approximately 10⁵-10⁸ equivalent CCID₅₀ in a single dosage unit. The volume of one single dosage unit can be between 0.2 ml and 5.0 ml and advantageously between 0.5 ml and 2.0 ml and more advantageously about 2.0 ml. One or more administrations can be done; e.g. with two injections at 2-4 weeks interval, and advantageously with a boost about 3 weeks after the first injection.

It should be understood by one of skill in the art that the disclosure herein is provided by way of example and the present invention is not limited thereto. From the disclosure herein and the knowledge in the art, the skilled artisan can determine the number of administrations, the administration route, and the doses to be used for each injection protocol, without any undue experimentation.

One embodiment of the invention is a method of eliciting an immune response in an animal, comprising administering a formulation for delivery and expression of a recombinant vaccine in an effective amount for eliciting an immune response. Still another embodiment of the invention is a method of inducing an immunological or protective response in an animal, comprising administering to the animal an effective amount of a formulation for delivery and expression of a genetic adjuvant as well as an antigen, epitope, immunogen, peptide or polypeptide of interest wherein the formulation comprises recombinant vaccine and a pharmaceutically acceptable carrier, vehicle or excipient.

The invention relates to a method to elicit, induce or stimulate the immune response of an animal, advantageously a human.

Another embodiment of the invention is a kit for performing a method of inducing an immunological or protective response in an animal comprising a recombinant vaccine and instructions for performing the method of delivery in an effective amount for eliciting an immune response in the animal.

The invention will now be further described by way of the following non-limiting examples.

### EXAMPLES

### Example 1

### Adjuvantation of AAV Vectors with Molecular Adjuvants

The following molecular adjuvants: IL-2, IL-12, Flagellin minimal TLR4 binding domain, CTA1-DD, and a version of fas antigen is cloned and expressed in an AAV1 vector which co-expresses SIV gag antigen. In the case of flagellin, a fusion construct is expressed between flagellin and SIV gag antigen. Each construct is evaluated for expression, adjuvant function, vector production and immune responses in mice. Vectors are further characterized for immunogenicity and efficacy in nonhuman primates, when given in combination with an AAV1 vector expressing SIV env antigen. IL-2 and CTA1-DD sequences are optimized and the vector expressing IL-2 is constructed and characterized

### Example 2

### Experimental Summary for the Assessment of Adjuvants in the Non-Human Primate (NHP) Model

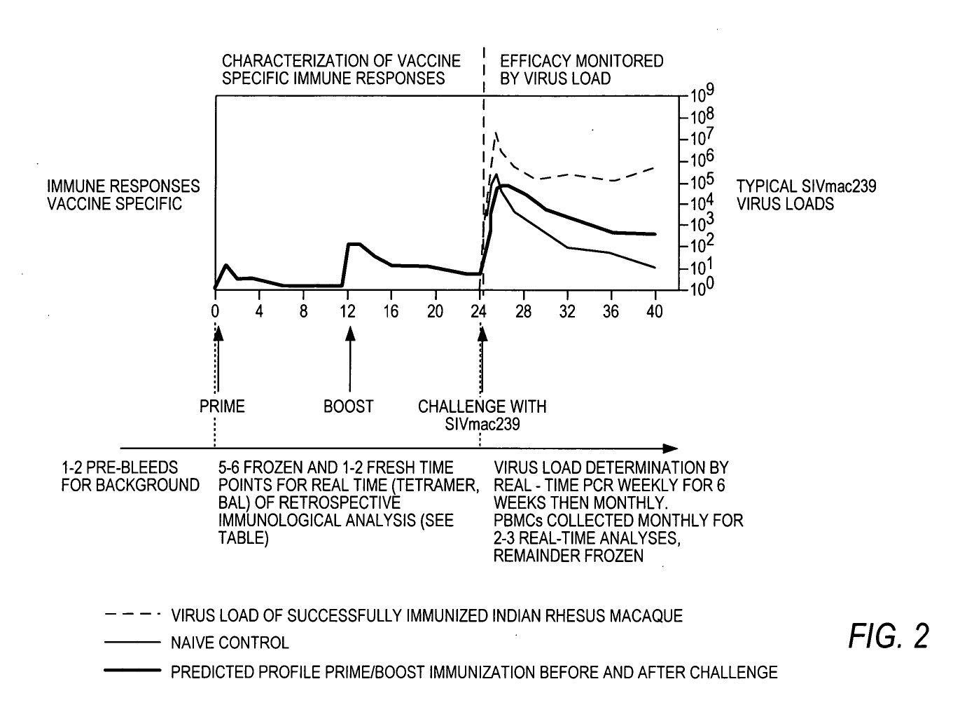

Immunological analyses upon pre-challenge time points (FIG. 2) for immunogenicity and characterization focuses upon the analysis of the breadth, specificity, memory phenotype and function of the immune responses elicited by the various vectors.

Time points are available for fresh analyses such as phenotype, tetramer (A*01 . . . etc.) and BAL analyses. Vials are frozen at most pre-challenge time points for archive and retrospective analyses.

The preferred challenge model for macaques is a repeated low dose mucosal challenge. Efficacy is assessed by virus load analyses and low level monitoring of specific vaccine generated responses.

TABLE 1
Study design

| | Mamu-A*01 | Non-Mamu-A*01 |
|---|---|---|
| Adjuvant #1 | 4 | 8 |
| Adjuvant #2 | 4 | 8 |
| Vector sans adjuvant | 4 | 8 |
| DNA/Ad5 | 3 | 3 |
| SIVΔNef | 3 | 3 |
| Naïve | 3 | 3 |
| (or empty vector controls) | | |
| Totals | 21 | 33 |

54

(**subject to statistical verification)

### Example 3

### AAV Adjuvanted Vector Design

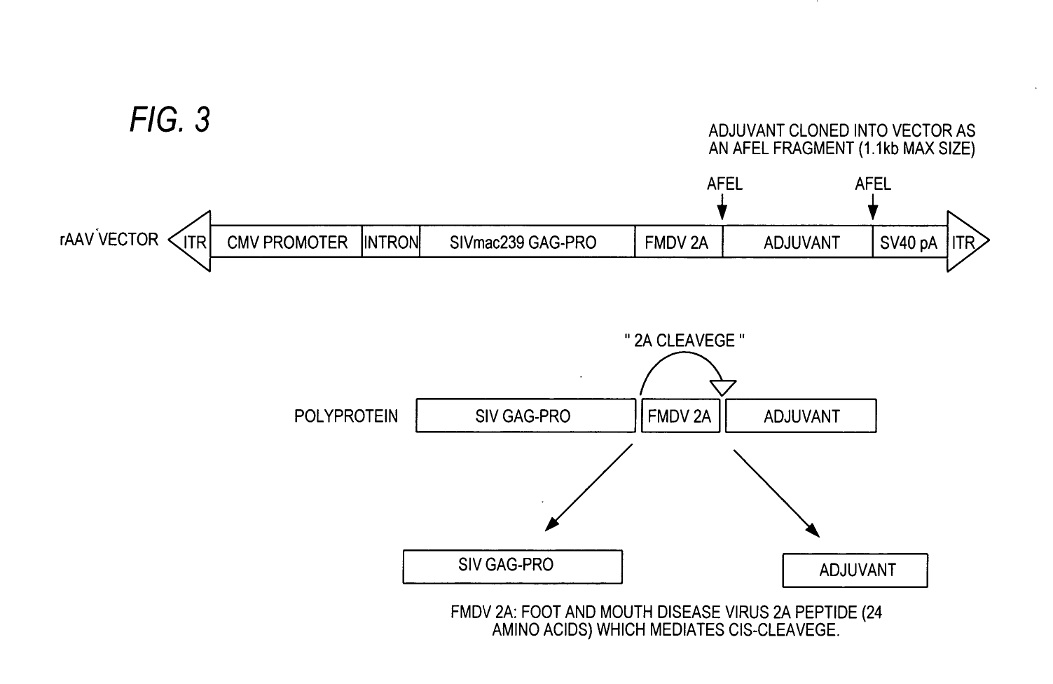

A prototype for a rAAV vector expressing SIV gag-pro is presented in FIG. 3. A molecular adjuvant is cloned in to the vector as an Afe1 fragment with a maximum 1.1 kb size. Larger adjuvants can be accommodated if the CMV cassette is truncated. The resultant polyprotein expressed by the rAAV vector comprises SIV gag-pro, a foot and mouth disease (FMDV) virus 2A peptide and a molecular adjuvant.

The FMDV 2A peptide mediates cis-cleavage of the SIV gag-pro and molecular adjuvant (see, e.g., Furler et al., Gene Ther. 2004 June ;8(11):864-73). FMDV 2A proteases are also disclosed in, for example, U.S. Pat. Nos. 6,896,881; 6,893,866; 6,884,623; 6,632,800; 6,586,411; 6,531,136; 6,232,099 and 6,171,592.

### Example 4

### Expression of Interleukin Adjuvants in an AAV Vector

The IL-12 construct includes a shortened CMV promoter, so that it can be accommodated in the AAV vector. The IL-12 adjuvant is about 1743 bp, however, with a shortened CMV promoter, it can be accommodated in the AAV vector.

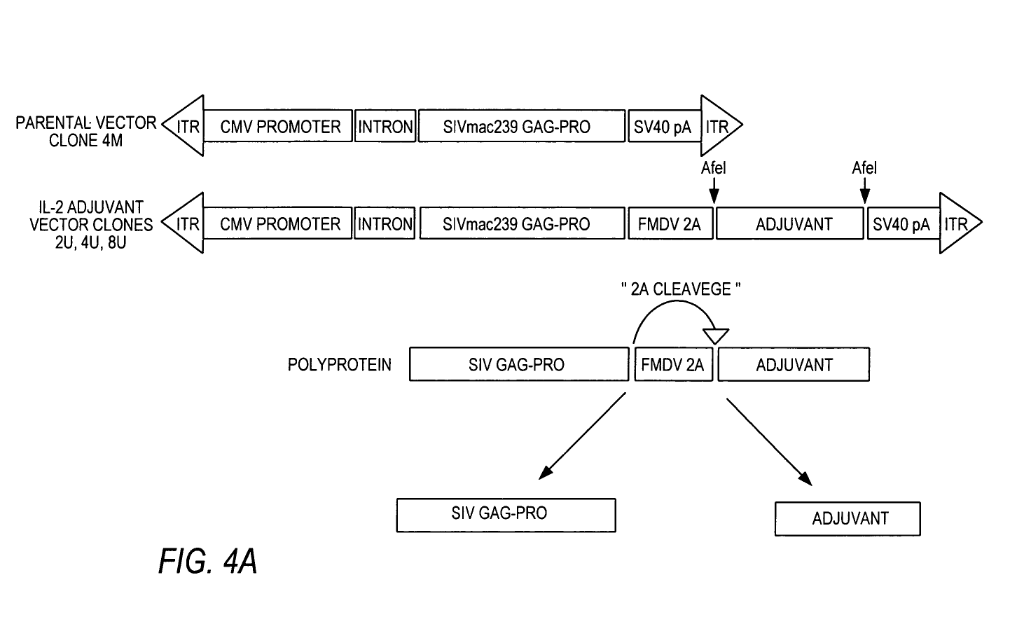

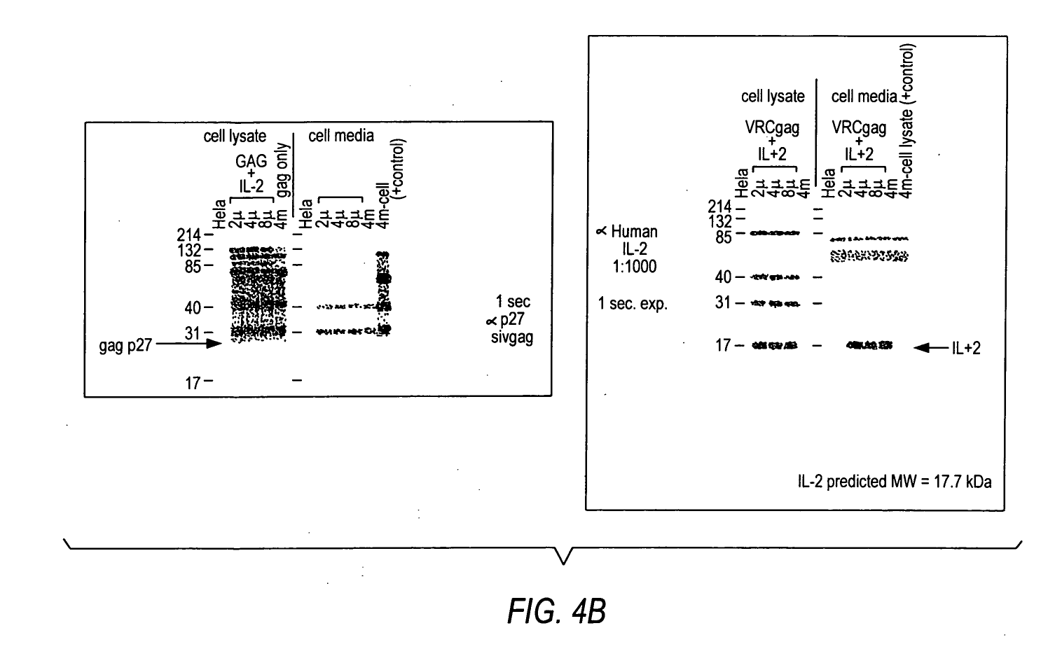

A parental vector clone expressing SIV gag-pro, as well as clones expressing genetic adjuvant IL-2, is presented in FIG. 4A. The resultant polyprotein expressing SIV gag-pro and Rhesus IL-2 is also presented in FIG. 4A. The IL-12 constructs include a shortened CMV promoter, so that it can be accommodated in the AAV vector.

Western blots demonstrating the expression of SIV gag-pro and IL-2 are presented in FIG. 4B.

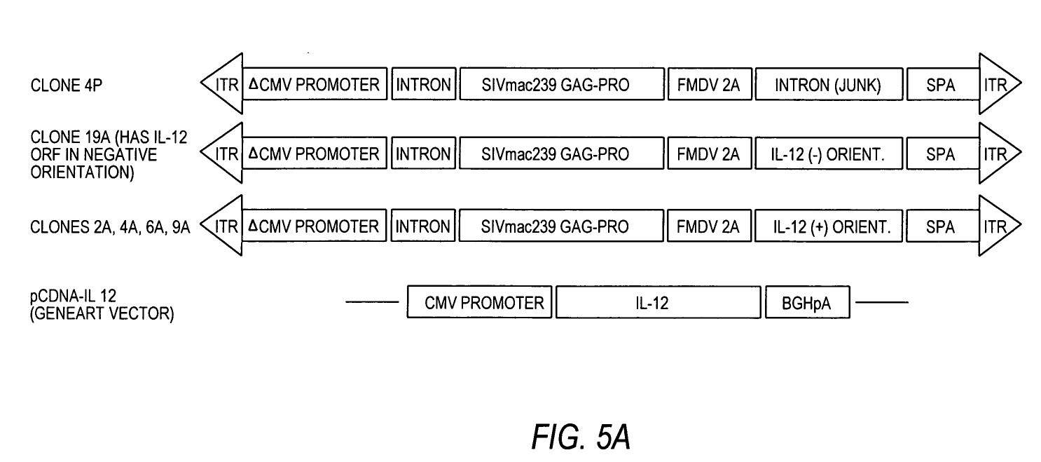

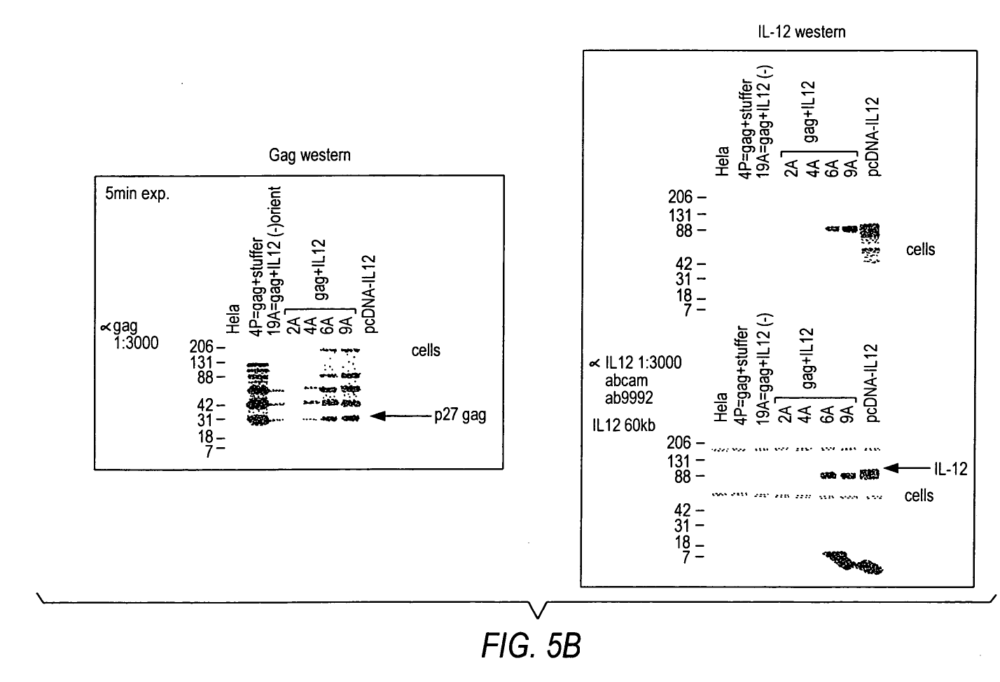

Transient transfection of SIV adjuvant constructs containing rhesus IL-12 is presented in FIGS. 5A and 5B. The constructs are presented in FIG. 5A and western blots demonstrating gag and IL-12 expression are presented in FIG. 5B.

### Example 5

### Expression of Flagellin Adjuvant in an AAV Vector

The flagellin constructs do not have the FMDV 2A cleavage site, because a fusion protein is being made between the SIV/HIV sequences and the flagellin sequences. Additional flagellin constructs without the SIV pro gene are also contemplated.

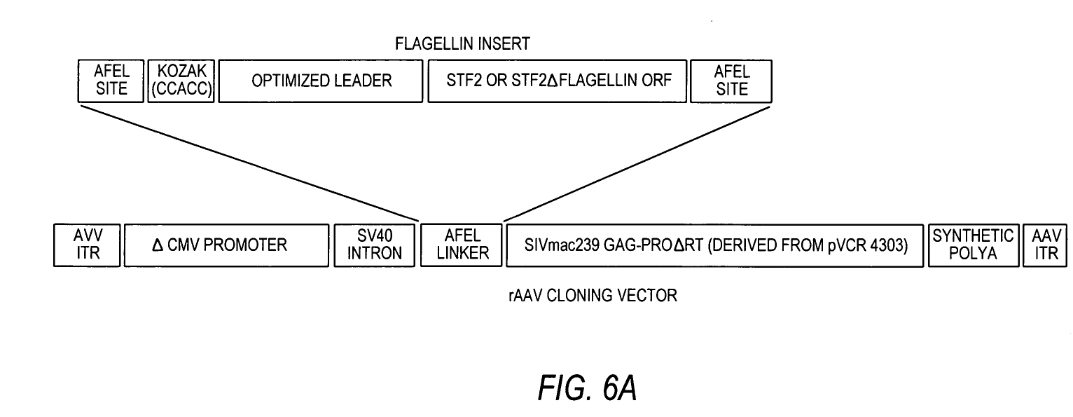

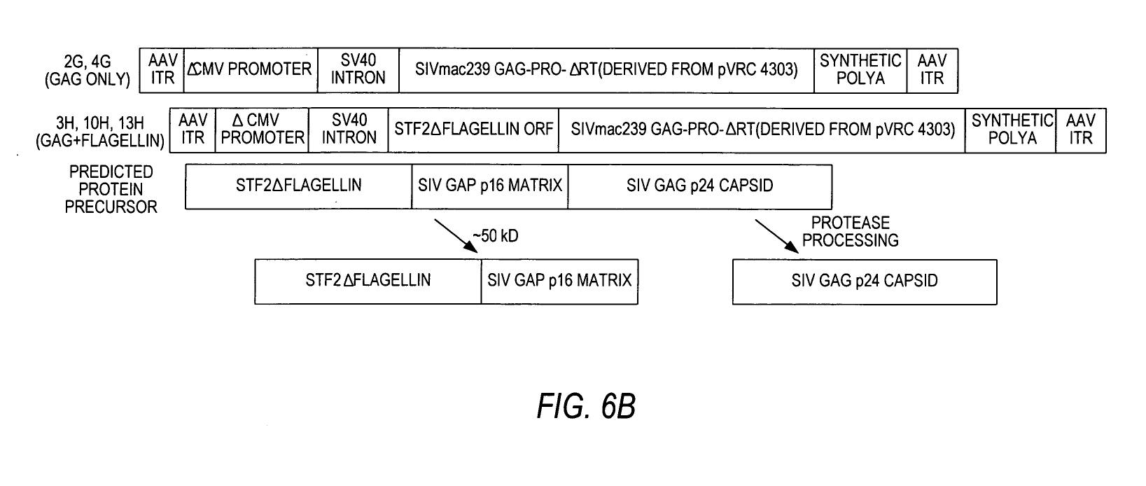

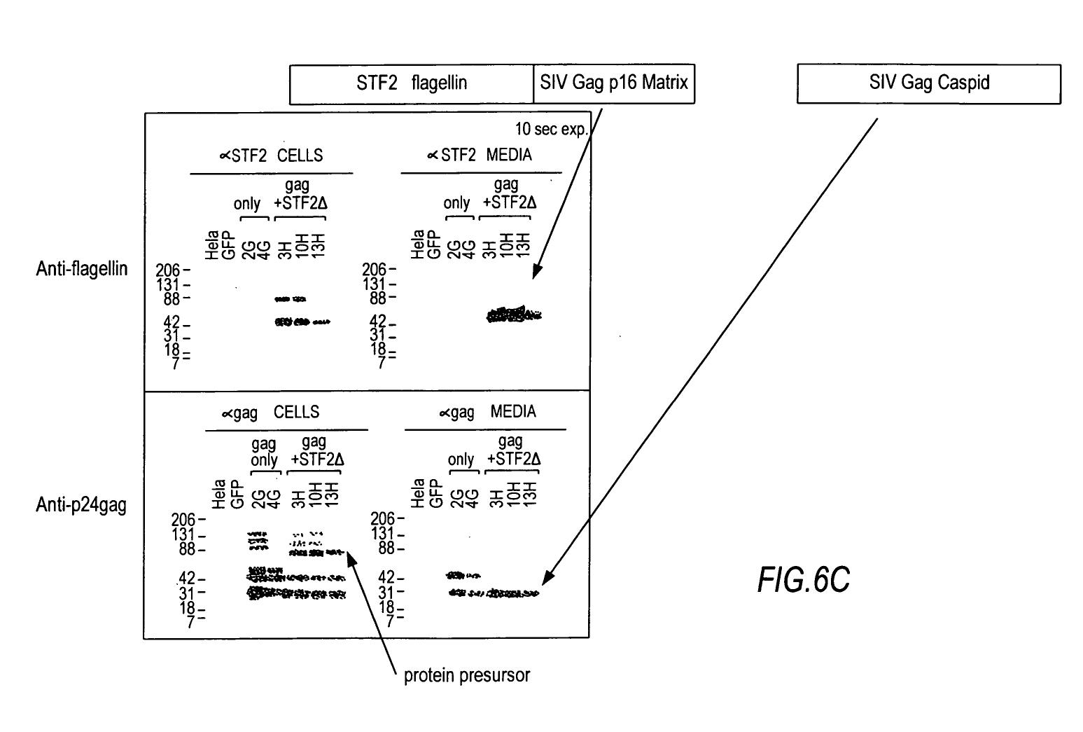

A cloning strategy for flagellin-SIVgag-pro fusion vectors is presented in FIG. 6A. The generated clones and predicted precursor proteins are presented in FIG. 6B. Western blots demonstrating gag and flagellin expression are presented in FIG. 6C.

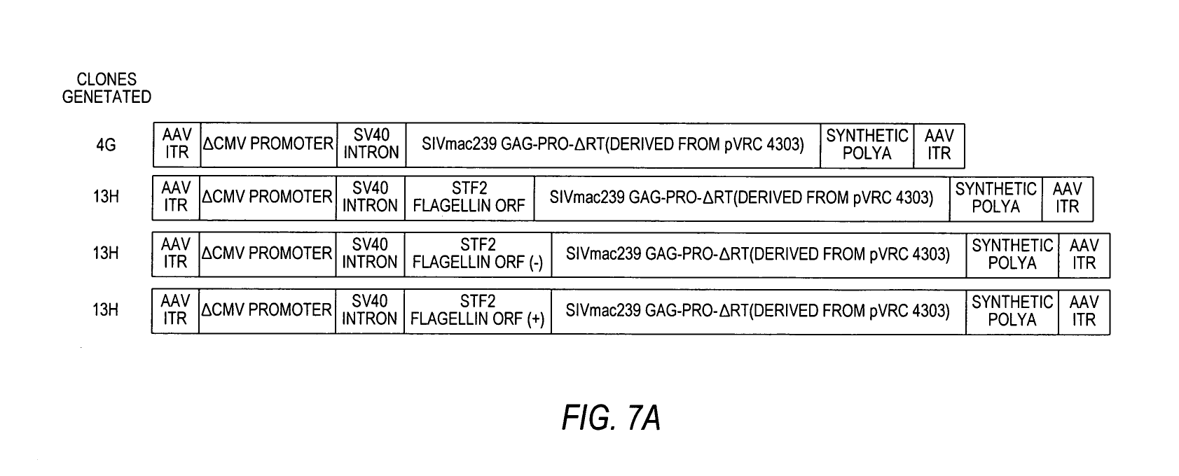

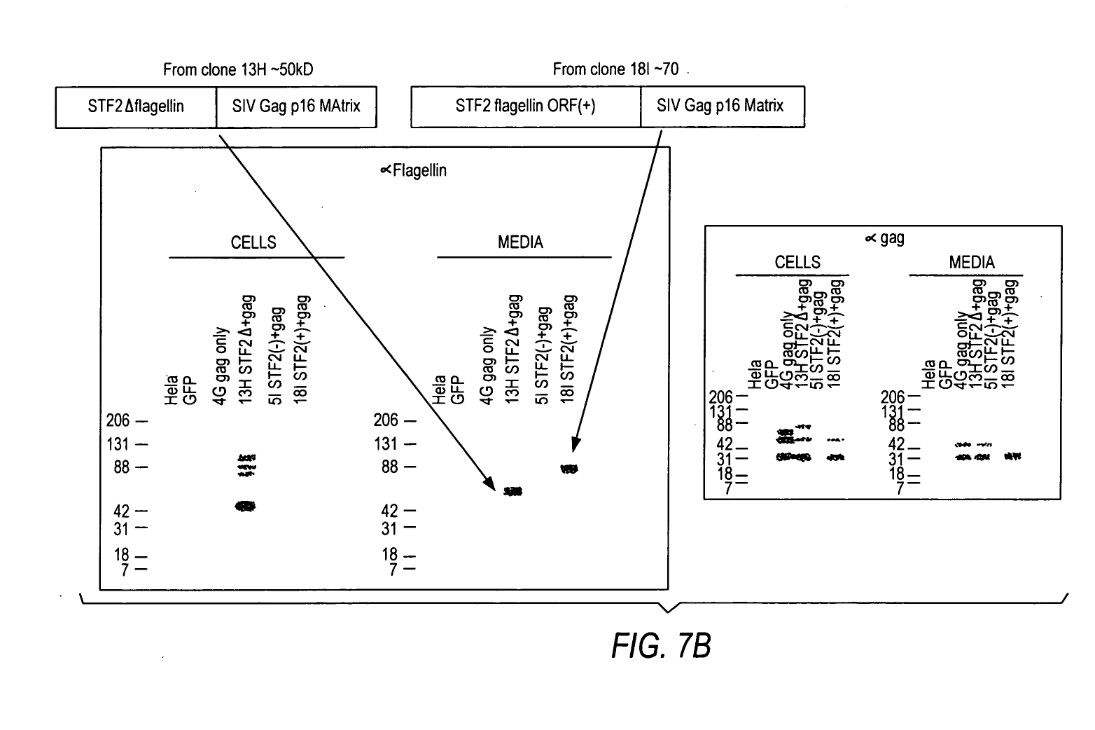

Additional flagellin constructs are presented in FIG. 7A. Western blots demonstrating gag and flagellin expression in transfected cells are presented in FIG. 7B.

### Example 5

### Expression of CTA1-DD Adjuvant in an AAV Vector

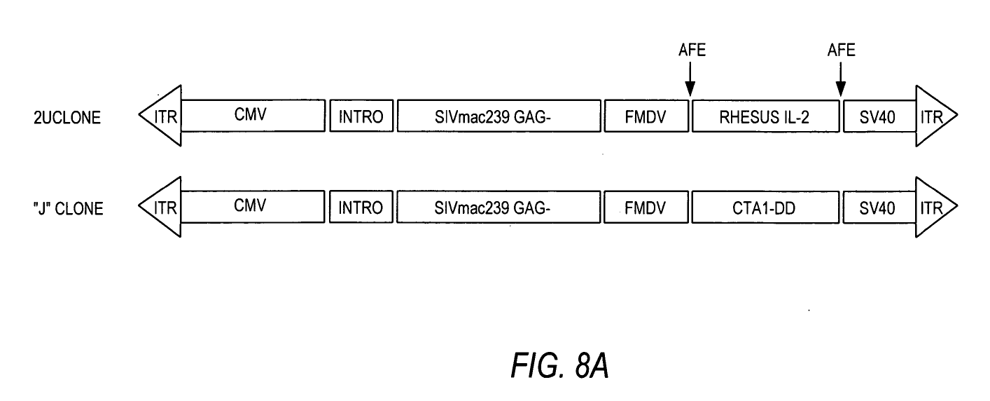

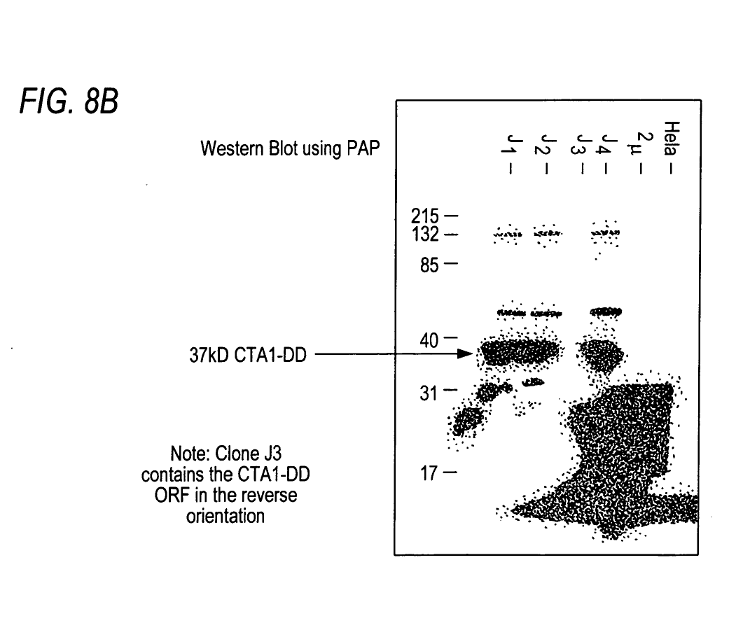

CTA1-DD expression in vitro is presented in FIGS. 8A and 8B. CTA1-DD constructs are presented in FIG. 8A and a western blot demonstrating CTA1-DD expression is presented in FIG. 8B.

The invention is further described by the following numbered paragraphs:

1. An immunogenic or vaccine composition comprising a viral vector, wherein the vector comprises a polynucleotide sequence encoding a genetic adjuvant.

2. The composition of paragraph 1 wherein the viral vector is an AAV vector.

3. The composition of paragraph 1 or 2 wherein the genetic adjuvant is a CTA1-DD, fas ligand, flagellin, IL-2 or IL-12.

4. The composition of any one of paragraphs 1 to 3 wherein the viral vector further comprises a polynucleotide sequence encoding an antigen, epitope, immunogen, peptide or polypeptide of interest.

5. The composition of paragraph 4 wherein the antigen, epitope, immunogen, peptide or polypeptide of interest is an immunodeficiency virus antigen, epitope, immunogen, peptide or polypeptide of interest.

6. The composition of paragraph 5 wherein the immunodeficiency virus antigen, epitope, immunogen, peptide or polypeptide of interest is an HIV or SIV antigen, epitope, immunogen, peptide or polypeptide of interest.

7. A formulation for delivery and expression of an antigen, epitope, immunogen, peptide or polypeptide of interest, wherein the formulation comprises the composition of any one of paragraphs 1-6 and a pharmaceutically acceptable carrier, vehicle or excipient.

8. A formulation for delivery and expression of an antigen, epitope, immunogen, peptide or polypeptide of interest, wherein the formulation comprises the composition of any one of paragraphs 1-3, a vector comprising a polynucleotide sequence encoding an antigen, epitope, immunogen, peptide or polypeptide of interest and a pharmaceutically acceptable carrier, vehicle or excipient.

9. The formulation of paragraph 8, wherein the antigen, epitope, immunogen, peptide or polypeptide of interest is an immunodeficiency virus antigen, epitope, immunogen, peptide or polypeptide of interest.

10. The formulation of paragraph 9, wherein the immunodeficiency virus antigen, epitope, immunogen, peptide or polypeptide of interest is an HIV or SIV antigen, epitope, immunogen, peptide or polypeptide of interest.

11. A method of stimulating an immune response in an animal comprising administering an effective amount of the formulation of any one of paragraphs 7 to 10 to cells of the animal and expressing the antigen, epitope, immunogen, peptide or polypeptide of interest in the cells.

12. A method of eliciting an immune response in an animal comprising administering an effective amount of the formulation of any one of paragraphs 7 to 10 to cells of the animal and expressing the antigen, epitope, immunogen, peptide or polypeptide of interest in the cells.

13. The method of paragraph 11 or 12 wherein the animal is a human.

14. A kit for performing any one of the methods of paragraphs 11 to 13 comprising the composition or formulation of any one of paragraphs 1 to 10 and instructions for performing the method of any one of paragraphs 11 to 13.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An immunogenic or vaccine composition comprising a viral vector, wherein the vector comprises a polynucleotide sequence encoding a genetic adjuvant and a polynucleotide sequence encoding an antigen, epitope, immunogen, peptide or polypeptide of interest

2. The composition of claim 1 wherein the viral vector is an AAV vector.

3. The composition of claim 1 wherein the genetic adjuvant is CTA1-DD, fas ligand, flagellin, IL-2 or IL-12.

4. The composition of claim 1 wherein the antigen, epitope, immunogen, peptide or polypeptide of interest is an immunodeficiency virus antigen, epitope, immunogen, peptide or polypeptide of interest.

5. The composition of claim 4 wherein the immunodeficiency virus antigen, epitope, immunogen, peptide or polypeptide of interest is an HIV or SIV antigen, epitope, immunogen, peptide or polypeptide of interest.

6. A formulation for delivery and expression of an antigen, epitope, immunogen, peptide or polypeptide of interest, wherein the formulation comprises the composition of claim 1 and a pharmaceutically acceptable carrier, vehicle or excipient.

7. A formulation for delivery and expression of an antigen, epitope, immunogen, peptide or polypeptide of interest, wherein the formulation comprises the composition of claim 2 and a pharmaceutically acceptable carrier, vehicle or excipient.

8. A formulation for delivery and expression of an antigen, epitope, immunogen, peptide or polypeptide of interest, wherein the formulation comprises the composition of claim 3 and a pharmaceutically acceptable carrier, vehicle or excipient.

9. A formulation for delivery and expression of an antigen, epitope, immunogen, peptide or polypeptide of interest, wherein the formulation comprises the composition of claim 4 and a pharmaceutically acceptable carrier, vehicle or excipient.

10. A formulation for delivery and expression of an antigen, epitope, immunogen, peptide or polypeptide of interest, wherein the formulation comprises the composition of claim 5 and a pharmaceutically acceptable carrier, vehicle or excipient.

11. A method of stimulating an immune response in an animal comprising administering an effective amount of the formulation of claim 6 to cells of the animal and expressing the antigen, epitope, immunogen, peptide or polypeptide of interest in the cells.

12. A method of stimulating an immune response in an animal comprising administering an effective amount of the formulation of claim 7 to cells of the animal and expressing the antigen, epitope, immunogen, peptide or polypeptide of interest in the cells.

13. A method of stimulating an immune response in an animal comprising administering an effective amount of the formulation of claim 8 to cells of the animal and expressing the antigen, epitope, immunogen, peptide or polypeptide of interest in the cells.

14. A method of stimulating an immune response in an animal comprising administering an effective amount of the formulation of claim 9 to cells of the animal and expressing the antigen, epitope, immunogen, peptide or polypeptide of interest in the cells.

15. A method of stimulating an immune response in an animal comprising administering an effective amount of the formulation of claim 10 to cells of the animal and expressing the antigen, epitope, immunogen, peptide or polypeptide of interest in the cells.

16. A method of eliciting an immune response in an animal comprising administering an effective amount of the formulation of claim 6 to cells of the animal and expressing the antigen, epitope, immunogen, peptide or polypeptide of interest in the cells.

17. A method of eliciting an immune response in an animal comprising administering an effective amount of the formulation of claim 7 to cells of the animal and expressing the antigen, epitope, immunogen, peptide or polypeptide of interest in the cells.

18. A method of eliciting an immune response in an animal comprising administering an effective amount of the formulation of claim 8 to cells of the animal and expressing the antigen, epitope, immunogen, peptide or polypeptide of interest in the cells.

19. A method of eliciting an immune response in an animal comprising administering an effective amount of the formulation of claim 9 to cells of the animal and expressing the antigen, epitope, immunogen, peptide or polypeptide of interest in the cells.

20. A method of eliciting an immune response in an animal comprising administering an effective amount of the formulation of claim 10 to cells of the animal and expressing the antigen, epitope, immunogen, peptide or polypeptide of interest in the cells.
